SEQUENCE LISTING (16 sequences)

SEQ ID NO: 1 - RNA, 260 nt, synthetic construct
  source 1..260: mol_type = other RNA; note = can be either dsRNA or ssRNA
ctgtcagccggatcaattcttgcactaacattctctttaactatgtcataaaacgcatactttccaattttaatcatcaaattgctcaacatagcaaaaacagcttctagtttgaagtgaggctgttttgcaggaatggaaattccaaacataatttttttgttgaaattggtaaatggataattcattgctgcaaaaaaagtagatgaaagagaagaaaacaagcagcaaaggagtgtaacaagcgcaaatgcaaaaac

SEQ ID NO: 2 - RNA, 259 nt, synthetic construct
  source 1..259: mol_type = other RNA; note = can be either dsRNA or ssRNA
ctgatcccatccctacaaggaaaatcaatgatactgctgaaaaatatcttacatcatttttgttcaaaatactcgtcatcaaaataaaataagctacgcaaaataactttcctgtttttggcattatcccatcaaaaatgcctttataaaaaaccttattttgtggatctattttgcttgaaacactttctttaataatgtcataaaaagaatacttgcttacctttgctgcgatgtttaatgcacaaataagcgctga

SEQ ID NO: 3 - RNA, 250 nt, synthetic construct
  source 1..250: mol_type = other RNA; note = can be either dsRNA or ssRNA
ttgcggttgaatttatgcagattataaatcttgaggttgatattttaccaaaaaagatatcagtttcttacagttttttgaaagatgaaggcatttcaattgacattgaagcagtcggaggaacaactaaaagagagattgtatcttgttcacatatagaaaatatgttagaaagattttttaatggttattcagattatggatatgtctttaataatctcgtctataatgacaatccagcacttgcaat

SEQ ID NO: 4 - RNA, 252 nt, synthetic construct
  source 1..252: mol_type = other RNA; note = can be either dsRNA or ssRNA
agtcatgctaggggtaacgatgggattaacaatttcatgcgctacttccactccaaattcagcacaggcacttggttcaactatttcaattttgtttattttatttagtggtgcatttaacaatcctaatgaatttccaagttggttaagatggataatttacatttcgcctatacaatatgcatttagagggctactacagtctcaggcaaagggaagagaattttcagatgggattggcaatatagatgg

SEQ ID NO: 5 - RNA, 250 nt, synthetic construct
  source 1..250: mol_type = other RNA; note = can be either dsRNA or ssRNA
atatgatcaattcacagggtaagcctaacgaaaagaaggacattgcaaaggattgcaggccatgcagcgactttgctaacaccacagaaggtgcatttgcagagtgcaagaacacatgcgatccaatgtctatgattgtcaatgttagcgatagcatccacacagtacacaggccaaccaccttaccatctggtaaagatgaccataaaaagccaactccatctgaaagtgaaataaacaatgaaacatt

SEQ ID NO: 6 - RNA, 236 nt, synthetic construct
  source 1..236: mol_type = other RNA; note = can be either dsRNA or ssRNA
tgatcatcaacgcaaaactgtaaataacctaagatatgatttagaagaaatattgcaatcaaacatttataaagaagatcaaaaagaaaatttagaaaaaaaattaggagaaacatctgaaaaaacactagtagaaatggatgaatttatgcatttaagtatgataaatggagtaatcaagaaaattgcaaagacacatcgtgaattttgcaaaaaggcaggagatcaccttgaaa

SEQ ID NO: 7 - RNA, 250 nt, synthetic construct
  source 1..250: mol_type = other RNA; note = can be either dsRNA or ssRNA
tgcgattattgaacctactgcatttcctattgaagcagatggagaacatattgaaactgtacatcctaagttaattgaaaaaaatacttctgctaaaagaattaaaataaatttcaaataagaaataaatgtagagtataaaccactacaaaaatatattacacttaaaaatggaatttcaaaaacaatgacaaaacacatttcagtacagattttaactgtataagctgcaaaaacatcataatatcca

SEQ ID NO: 8 - RNA, 250 nt, synthetic construct
  source 1..250: mol_type = other RNA; note = can be either dsRNA or ssRNA
cgccctaacaatttcacagtctacagtggataaatataacaagaattatcgtttgggaagaagttgttttaatattattccgagcacaactggagcatcagatgctatagaatgtgttttaccagaaatgaaaaataaaataaaaggaattgcaacaagagtgcctgtaattgatgtttcaatggttgatttaaacttattgcttaaaaaagaagtggaaaacattgaagaaatttgcaaagaaatccaa

SEQ ID NO: 9 - DNA, 749 bp, Enterocytozoon hepatopenaei
  source 1..749: mol_type = genomic DNA
tgttattttctacaatattattaaatttcattagagcagaggatgagaagattagtgaaattcacagtaagaacaagggaacagcagtatctcacacagtatttggctctaacattgagttaaaccaagcatttgatggaaagaatgatttgactaacattagcgcatttagtgcaaaagaaaaaggagaaaaggcagcagatcttactttgaagacagggactgacatcttaataaacaaccaagaagtttatgatttcttaaagaatagcagcagcgtaaaggacgaattagcttctttggatagcaaagaaaaatttgaggctcttaaaaaggcagtagaaaaaaacaaaaaggaactccctgctgcggtagtaagttttataagtggttatgatgcatcaaaagtagctgattttgaatctgaatttttgggctcaaaaactattaaaccatgcgttatggaatcaggtcttaaaggagcacttactgacaagaaaggtgtaatgtttatttctttttctttaaacaataaggaatacaaaacaggcttaattgaaattactgaaaaggatggaaaattggaaattgctggttcttcatcagttagtggcttaaaagtatttggaattattgtgttagttttggtattagtatcattggttcttattgctggttactacttttatttgcaacaaaagaagaaatctgcagaaggattaaatgcagatgattcaatgcaaccttaa

SEQ ID NO: 10 - DNA, 779 bp, Enterocytozoon hepatopenaei
  source 1..779: mol_type = genomic DNA
tatgaaaccatttggcttgtttttgaatatctttgcattattttttatgttaatagtcaaggcagaagatggatgtatcgaagaaagcaaaacctatgttattaacatcaaggcccatgttgaaggtttgattcaacaatctattatttcactgtctgcaaagtttgaagattctatacttacaaatgaatatgttgctgaaaataaggaatccgctaaagttgcagctacccgatcatatctcggtgttatttttcaagagttaaacgaagacctttttaagtataaagtgcaagtcaatcttgttttggacccacaagaaatagaacaggtggaccacattggaaaggtagatccttcatgtgaaattggaaatgcgcttaagtcaagaacatcagctgcttttgacatgttgtctgaaaaatatggtaaaaatgttggcaaccacctttatcttgtaggatgtccaatatcgcaggatgataagtctccaattgaagtttctgaaaagaatgcgtgtggtagggtaataggtgttatgtggtcaggctcagcaaaaacaaaagacttgataaagtctgcaattatacaggcactgacaggaacaccaaatacatataaagatggtttgttggaaatggctgataaaaataatttgtgcagatactgtgaaaggtgcatttcaagcaatgaaagtgctcttggtattttactgaaatatttaacaaaagttacatttttaaatgatgaataaatttagttaattttaaaaaaaaaaa

SEQ ID NO: 11 - DNA, 1422 bp, Enterocytozoon hepatopenaei
  source 1..1422: mol_type = genomic DNA
ttagctgaacgaataattgataaccagtgcattttttacagaaaacttagccaattctgtttcaatttctttgatcgtcttctcaattaagaaatatgcagctagttgttcaagctggtgattaaattctgcctctgtgagttctttatcacaatcagtgttcatttgatcatgcatttcatttttaatggtggatgtttctgtttcaatgtttgttaaatcagtctgtaatgaagtaagctgattttcagcttgtgttttatttgctattaatacatccttttctgttgttttttcatttaactcttcttctaatgaatcaatttgttcttcaatgcctgtcttttgttcgtttaacttttcaatttcagctttatttgcctcaatatctgcatcaagatttgcttgttcagtatcaatattattttgtagtgtatcaatttcttcttgtgtcgtagattcagccccagctggcaccaaattaccttctaggctagcaatttcatcatcaattgcggcattttcatcttcaagtttagttttttcatctgttttagtttggatttgcttgtttatttcatcaatttctgcctgtttggcattaatttgctcttgttttggatccaattcagctgtttttgttgttatttgaccttctaaatttgttattgcagtgcttaagtcatttatgtgagtattaagactttcctgggtgttaaaagtattttctcctattgttacaggataactttcaggttgtgttccattaaaaaatttttctgcttctgttttttctgtgtttttagcatccaattcagtatttaaatcagctatatcaccttgtattgttgccaattcattatttaaagtttctaactctgtatctttttgagacaaatcagttgttaatgcttctaaatctgtatcaatttgatctattgaagccaaattagtgttttttaattcttcttttgcatttatttgttcatgaatatcgttgttttttaatttttgttctaaatcttctttatcactgtcaatattttcttgtcgttctgtaaaatcagtctgcttttctgtttcagtcgttatatcttccgtaacagctttcaaattatcattagcatcggcaagtgcattttcaatcggtgtaatctctccttctattgatgtaatttgttcatctatttttgttatatcagattctttttcttctatttgtttttcaagatctttaatttgttgctctaaatcttgtagttgtttataatcatcagtatacattgttttgttttcaaaaatttctggttcaatttcatttttggtttcttcaagtttattgttggctatttccaattcctcttgtaattcagcttctgttttccttgtagctaaactcaataaaaagtaatttaaaatcat

SEQ ID NO: 12 - DNA, 1866 bp, Enterocytozoon hepatopenaei
  source 1..1866: mol_type = genomic DNA
ttaagtggtataacatcctgcctcatctgctactttttctatattcattgcttttgccatttttgtataaatatcatatcttttctttgatttatcaatattattgttatttttacactcaagttctccgtttattgccttagtggagtatccaaaatggtcaggaccaaccccttcttttcccttaacatttttatcccaaaaccactgggaaacatcatatgccttttgcgtatccttagcaactttatcaggatcatcgtatagctcgctgttaccgtacagatcagcagaagcatttttgtagttatcaggccatgatagctgcataaatccccttccataatattgcttacccggtgcaccctggtccgatccatattgtccagggcacgtgttgccagcacatgcaatttcttcaacgtactgaaagcctccgctctcgtgcgcaatttgcgccagaaactgagcagccatgtctttatccttccagttcttgttaacaccatctacaacagcatttaagtattcttcgttaggtgaaaagttacattccttcataacttgcattaaagtatctttatcaatgatattaccaccattactttgattaccttttgaatcttttgaatcttttgaatcacttgatttattgctattgctattgctagtatttttatttttatcattttgagtattattagaattattaccagatgatgatgcattattggaattattaccagaagattgtgaagaagaagatggtgatgtatttgatgatgaattactactagatggtttagaattacctgaagatgatgaagagggtgtattagatgaaccactacctgatgaagtatttccactgttattaccacttgaagaattaccacttgaagaattaccacttgaagaattattgcttgaagaattaccacttgaagaattattgcttgaagaattaccacttgaagaattattgcttgaagatggatttgaactattttctgtactgctactgccacctgatgaattaccactgctactgccacctgatgaaaatgaccctgttgatgatacttggcctggtcctgtatactccaactttatttctctgggtgctgttttctctgttattgtttgggtcactgttttttcctttgccttgcttatcatttctgccagtgtttttaacaggctgtcgtttgctggtgaatctgcttttgttgatatcgatggttgcatagtaccaatgccataattattaggctgaccactttctgttttattttcatttttattctggtgattacttttatttgaattgctttctgtttttgtaggttccttttgtttggttactgttactgtttttttgtgtttgtatactgtcttagttttgacattatctttcttattatgactattatgtttattttctttttttggttttttagttttactttttttaggtttatcctttttatttttgctttcatcttttatgttcataatactatcttcactattatcatttgaattcatttcatcctcatttgactctatatttgatggttttttctttttatcttttttacttttggtattaaacaaattatcaaaatcactcttttctcctgtttcgtcaatttcttcagattcttttttatttgtacatcctaatttaccatctgacatggaacaataagaattttctggacattttagtgaaatccatgatgcatccgcagaacaaaaacttaatgtgtgtttgtcagagcaaatagcagaccctggcaaacagctgtttttaccctttaaaaaacgtgcataaagcaaaaatgcggtaatgtttaacaacat

SEQ ID NO: 13 - RNA, 240 nt, synthetic construct
  source 1..240: mol_type = other RNA
gaatctgaatttttgggctcaaaaactattaaaccatgcgttatggaatcaggtcttaaaggagcacttactgacaagaaaggtgtaatgtttatttctttttctttaaacaataaggaatacaaaacaggcttaattgaaattactgaaaaggatggaaaattggaaattgctggttcttcatcagttagtggcttaaaagtatttggaattattgtgttagttttggtattagtatca

SEQ ID NO: 14 - RNA, 260 nt, synthetic construct
  source 1..260: mol_type = other RNA
gcttttgacatgttgtctgaaaaatatggtaaaaatgttggcaaccacctttatcttgtaggatgtccaatatcgcaggatgataagtctccaattgaagtttctgaaaagaatgcgtgtggtagggtaataggtgttatgtggtcaggctcagcaaaaacaaaagacttgataaagtctgcaattatacaggcactgacaggaacaccaaatacatataaagatggtttgttggaaatggctgataaaaataatttgtg

SEQ ID NO: 15 - RNA, 222 nt, synthetic construct
  source 1..222: mol_type = other RNA
acaaaaattaaaaaacaacgatattcatgaacaaataaatgcaaaagaagaattaaaaaacactaatttggcttcaatagatcaaattgatacagatttagaagcattaacaactgatttgtctcaaaaagatacagagttagaaactttaaataatgaattggcaacaatacaaggtgatatagctgatttaaatactgattggatgctaaaaacacagaa

SEQ ID NO: 16 - RNA, 226 nt, synthetic construct
  source 1..226: mol_type = other RNA
gatacgcaaaaggcatatgatgtttcccagtggttttgggataaaaatgttaagggaaaagaaggggttggtcctgaccattttggatactccactaaggcaataaacggagaacttgagtgtaaaaataacaataatattgataaatcaaagaaaagatatgatatttatacaaaaatggcaaaagcaatgaatatagaaaaagtagcagatgaggcaggatgtt

(19) United States
(12) Patent Application Publication
(10) Pub. No.: US 2026/0242795 A1
(43) Pub. Date: Aug. 20, 2026

(54) COMPOSITIONS AND METHODS FOR AQUATIC MICROSPORIDIAN INFECTION

(71) Applicant: ViAqua Therapeutics Ltd., Haifa (IL)

(72) Inventors: Shai UFAZ, Givat Ada (IL); Ami SCHLESINGER, Kibbutz MaAgan Michael (IL); Ori KOSHET, Kibbutz Kfar HaMaccabi (IL); Margarita KANTEEV, Tirat Carmel (IL); Svetlana FRIDMAN, Yokneam (IL)

(73) Assignee: ViAqua Therapeutics Ltd., Haifa (IL)

(21) Appl. No.: 19/143,942

(22) PCT Filed: Dec. 20, 2023
(86) PCT No.: PCT/IL2023/051289

§ 371 Date: Jun. 27, 2025

Related U.S. Application Data
(60) Provisional application No. 63/435,862, filed on Dec. 29, 2022.
(60) Provisional application No. 63/539,132, filed on Sep. 19, 2023.

(51) Int. Cl. C12N 15/113; A61K 47/36; A61P 31/10

(52) U.S. Cl. CPC C12N 15/1137; A61K 47/36; A61P 31/10; C12N 15/113; C12N 2320/30

(57) ABSTRACT

Chitosan-RNA particles comprising low molecular weight chitosan and ss or dsRNA partially complementary to, binding to or at least 90% identical to mRNA targets of microsporidian parasites pathogenic in farmed fish or crustaceans, compositions and farmed aquatic animals comprising the same, and methods for their use in treating or preventing microsporidian infection in aquaculture are provided.

### RELATED APPLICATION/S

This application claims the benefit of priority of U.S. Provisional Patent Application Nos. 63/435,862 filed on 29 Dec. 2022 and 63/539,132, filed 19 Sep. 2023, the contents of which are incorporated herein by reference in their entirety.

### SEQUENCE LISTING STATEMENT

The XML file, entitled 98402 Sequence Listing.xml, created on 23 Nov. 2023, comprising 24,576 bytes, submitted concurrently with the filing of this application is incorporated herein by reference.

### FIELD AND BACKGROUND OF THE INVENTION

Shrimp is one of the most valuable aquaculture species globally, and the most internationally traded seafood product. Consequently, shrimp aquaculture practices have received increasing attention due to their high value and levels of demand, and this has contributed to economic growth in many developing countries. The global production of shrimp reached approximately 6.5 million tons in 2019 and the shrimp aquaculture industry has consequently become a large-scale operation. However, the expansion of shrimp aquaculture has also been accompanied by various disease outbreaks, leading to large losses in shrimp production. Among the diseases, there are various viral and parasitic diseases which can cause serious damage when compared to bacterial and fungi-based illness. In addition, new microsporidic diseases occur rapidly, and existing diseases can evolve into new types.

Aquaculture farming, especially shrimp farms, is a rapidly growing industry as they provide an abundant, cheap source of protein. The global shrimp market size was USD 28.45 billion in 2020, and the market is projected to grow from USD 33.81 billion in 2021 to USD 53.63 billion in 2028 at an average growth rate of 6.81% during the 2021-2028 period. Of key concern to this market is aquaculture disease.

Enterocytozoon hepatopenaei microsporidian (EHP) is a microsporidian parasitic disease that affects aquacultures primarily in South and Southeast Asia but has been recorded in Venezuela as well. EHP severely restricts shrimp growth, causes size variability in the population, and increases shrimp susceptibility to acute hepatopancreatic necrosis disease (AHPND) and septic hepatopancreatic necrosis (SHPN).

Enterocytozoon hepatopenaei microsporidian (EHP) is an obligatory spore-forming parasite that replicates within the cytoplasm of the tubular epithelial cells of the hepatopancreas (HP) in shrimps. The EHP genome lacks a large set of mitochondrial genes, preventing ATP generation, making EHP dependent on host energy metabolism. EHP infection is initiated through spores that puncture the hepatopancreas cell plasma membrane and deliver their content into the cell cytoplasm where the sporoplasm matures into the spore. During this process, infected HP epithelial cells swell and eventually rupture to release mature spores, facilitating either infection of other HP cells or release of mature spores into the environment via the feces, thence infecting other shrimps. EHP is not associated with shrimp mortality, however, perhaps more critically from an economic standpoint, EHP infection causes growth retardation and severe production losses in farming practices.

Although EHP was first reported and characterized more than 10 years ago, there is still little known about the immune response of shrimps to EHP, and to date, there is no effective treatment against EHP besides efforts to avoid contamination.

### EHP and Microsporidian Genetics

Several Microsporidian genomes, including Enterocytozoon hepatopenaei (E. hepatopenaei, EHP), have been published to date (see, for example, Wiredu Boakye et al, Environ. Microb 2017, 19:2077-2089 and Bojko et al, Exp Suppl 2022:114:247-283). The sequence analysis of E. hepatopenaei revealed a very small and compacted genome of 3.5-megabase pairs, comprising of just a few thousand genes. The E. hepatopenaei genome lacks most of the introns and intergenic spacers usually found in eukaryotic genomes. The majority of the genes are also shorter than their corresponding homologues. In addition, in-depth analysis of the predicted genes showed absence of genes of some biosynthetic and metabolic pathways, while other such pathways included a relatively limited number of genes. Recent studies using the sequencing information have revealed some details of microsporidian evolution and metabolism such as homologues of bacterial ADP/ATP transporter suspected important in E. hepatopenaei energy metabolism.

Being an obligate parasite, E. hepatopenaei, and microsporidia in general, relies on its host to provide it with the energetic and metabolic needs. The compensation pathways, and their function, are poorly understood.

For many years, microsporidia were thought to be lacking the mitochondria, and accordingly proposed to have evolved before the appearance of the eukaryotic mitochondria. Enterocytozoa lack electron transport chain and Kreb's cycle.

Intracellular symbiotic organisms use mitochondrial carrier family proteins (MCF) in order to acquire various substrates, including ATP, from the host cell, in order to provide the energy for the protein transport and other necessary mitosomal activities. However, as the microsporidian genomes have apparently lost all of the genes for the MCF proteins, it is not known how the parasite acquires the necessary ATP for its function. Several bacterial intracellular parasites, such as Rickettsia, possess a nucleotide transporter which is used for ATP import from their eukaryotic host cell. However, the use of such bacterial-like nucleotide transporters to acquire ATP from a eukaryotic cell is unknown in a eukaryotic parasite. There are no homologues of these proteins in either vertebrate or invertebrate species sequenced to date.

### Gene Silencing

The process of post-transcriptional gene silencing is most likely a cellular defense mechanism used to prevent the expression of foreign genes, thought to be shared across kingdoms. The presence of long dsRNAs in cells stimulates the activity of a ribonuclease III enzyme referred to as Dicer, which is involved in the processing of the dsRNA into short pieces known as short interfering RNAs (siRNAs). These are typically about 21 to about 23 nucleotides in length and comprise about 19 base pair duplexes. The RNAi response also features an endonuclease complex, commonly referred to as an RNA-induced silencing complex (RISC), which mediates cleavage of single-stranded RNA having sequence complementarity to the antisense strand of the siRNA duplex. In some organisms, an amplification stage may follow the initiation stage of gene silencing, involving an RNA dependent RNA Polymerase (RdRP), which may subsequently lead to degradation of RNAs outside the initial dsRNA region of homology. It has been shown in some species that RNAi mediated interference spreads from the initial site of dsRNA delivery, producing interference phenotypes throughout the injected animal.

Microsporidia have been classified as Fungi, which have shown an evolutionarily diverse repertoire of silencing proteins. Some of these are distinct from vertebrate silencing homologues. In Trypanosoma brucei a DICER-like homologue was identified. RISC homologues have also been described, and RNAi-related transcripts have been identified in simple, parasitic eukaryotes such as Giardia and Trichomonas. However, the function of such enzymes and their products is unclear. Further, although DICER and RISC enzymes have been detected in some species of Trypanosomes, other Trypanosomes have been identified as RNAi-negative, consistent with the observation that many eukaryotic parasites are genetically heterogeneous where RNAi pathways are concerned (Wadi et al, 2020).

RNAi gene silencing in intracellular eukaryotic parasites has been demonstrated either for host proteins suspected of critical roles in the parasites life cycle (as in T. cruzi), or in free-living forms, such as the extra-cellular forms of Plasmodium and T. brucei that can be cultured in-vitro. In addition to the requirement of traversing at least one parasite membrane of undefined permeability and composition, additional obstacles to effective RNAi methodology for intracellular forms of eukaryotic parasites include the heterogeneity of RNAi pathways in lower, parasitic eukaryotes, poor understanding of the function of such pathways, and the limited knowledge of parasite metabolism, and therefore difficulty in selecting effective targets for silencing the parasite genes.

RNA interference (RNAi) is a powerful technique that has been widely applied to inhibit both DNA and RNA viruses in a variety of vertebrate and invertebrate hosts (see, for example, US Patent Publications US2005/0080032 to Gross et al; US2013/0315883, 2020/0032267 and 2020/0405782 to Sayre et al), including shrimp and other farmed aquatic species (Abo-Al-Ela et al, J Agric Food Chem 2021 69:4343-4355; Gong and Zhang, Dev Comp Immunol 2021 115:103907; US Patent Publication 2012/0108649 to Loy et al; Sarathi et al Mar Biotech 2008 10:242-249;), and economically important parasitic microsporidian (nosemosis) infection in bees (U.S. Pat. Nos. 9,932,579 and 8,822,426 to Paldi and Glick; Kim et al Arch Insect Biochem Physiol 2020 105:e21734; and Rodriguez-Garcia et al, J Exp Biol 2018 221:jeb184606) but no previous studies have demonstrated use of this technique for controlling EHP.

The present inventors have previously developed an oral platform for highly effective dsRNA delivery in farmed crustaceans, and in particular, for treatment and prevention of White Spot Syndrome Virus (WSSV) in shrimp (PCT Publication WO2017/187440 and U.S. Pat. No. 10,973,774). Other relevant publications include Itsathitphaisarn et al, J. Invert. Pathol, 2017, 147:76-85, US 20190175518 to Ufaz et al, US 2013/0245091 and U.S. Pat. No. 9,011,919 to Rozema et al, US 2014/0335192 to Ward et al, US 2011/0033547 to Kjems et al., PCT Publication No: WO 2008/003329 to Besenbacher et al, US Patent Publication 20080194504 to Kyle et al, EP Patent No. 2397123 to Aarhus Univ, US201020295355-A1 to Baker Shenda et al, US20110064664-A1 to Lopez-Berestein et al, US20120238735-A1 to McManus et al; Sarathi et al, Marine Biotech 2008 10.3:242-249 and Feroskhan et al, Current Nanoscience 2014, 10.3:453-464.

### SUMMARY OF THE INVENTION

According to an aspect of some embodiments of the present invention there is provided a composition comprising chitosan and at least one type of RNA molecule comprising a nucleic acid sequence at least 50 bases in length wherein the RNA molecule is capable of silencing expression of a gene of a microsporidian parasitic in a farmed aquatic animal when administered to said farmed aquatic animal.

According to an aspect of some embodiments of the present invention there is provided a method for producing a chitosan-RNA composition comprising RNA capable of silencing expression of a gene of a microsporidian parasitic in a farmed aquatic animal, the method comprising:
i) rapidly adding an RNA solution to an volume of 75-100% deacetylated low molecular weight chitosan solution, wherein the chitosan solution comprises 2-20 mg/ml low molecular weight chitosan in 0.01-0.1M organic acid,
ii) desalting and concentrating the chitosan-RNA complex solution, and
iii) drying the desalted, concentrated chitosan-RNA complex solution,
thereby producing the particulate chitosan-RNA composition.

According to some embodiments of the present invention the organic acid is citric acid.

According to some embodiments of the present invention the organic acid is 0.02 M.

According to some embodiments of the present invention the RNA solution for the method of producing the chitosan-RNA composition comprises 0.2-10 mg/ml RNA in water.

According to some embodiments of the present invention the chitosan-RNA particles are the compositions described in the invention.

According to an aspect of some embodiments of the present invention there is provided a nutriceutical composition comprising farmed crustacean food and the composition described herein.

According to an aspect of some embodiments of the present invention there is provided a farmed crustacean comprising the nutriceutical composition described herein.

According to an aspect of some embodiments of the present invention there is provided a farmed crustacean comprising the composition as described herein.

According to an aspect of some embodiments of the present invention there is provided a method of treatment or prevention of EHP infection in a farmed crustacean, the method comprising feeding the farmed crustacean the composition described herein or the nutriceutical composition described herein, wherein the at least one type of RNA molecule is targeted to an EHP gene product, and optionally wherein ingestion of the composition or nutriceutical by the farmed crustacean results in reduction in the level of the EHP parasite in the farmed crustacean, compared to the same farmed crustacean ingesting feed devoid of RNA targeted to an EHP gene product.

According to some embodiments of the present invention the microsporidian is Enterocytozoon hepatopenaei (EHP).

According to some embodiments of the present invention the at least one type of RNA molecule comprises one or more of the following:
(a) at least one sequence capable of binding through complementary base pairing to an EHP target mRNA molecule;
(b) at least one sequence having at least 90% sequence identity to an EHP target mRNA molecule;
(c) at least one sequence at least partially complementary to an EHP target mRNA molecule;
(d) at least one sequence identical to at least 21 contiguous bases of an EHP target mRNA molecule.

According to some embodiments of the present invention the partially complementary sequence or sequence capable of binding through complementary base pairing to the EHP target mRNA is complementary to a sequence of the EHP target mRNA at least 50, at least 75, at least 100, at least 150, at least 200, at least 250, at least 400, at least 500, at least 600, at least 750 bases in length.

According to some embodiments of the present invention the at least one RNA molecule comprising at least one sequence identical to at least 21 contiguous bases of an EHP target mRNA molecule is identical to at least 50, at least 75, at least 100, at least 150, at least 200, at least 250, at least 400, at least 500, at least 600, at least 750 bases of the EHP target mRNA molecule.

According to some embodiments of the present invention the at least one type of RNA molecule is a single stranded RNA (ssRNA).

According to some embodiments of the present invention the at least one type of RNA molecule is a double-stranded RNA (dsRNA).

According to some embodiments of the present invention the EHP gene is selected from the group consisting of the EHP polar tube protein 2 (PTP-2) gene, the EHP Heparin binding protein (EhSWP1) gene, the EHP ATP binding cassette (ABCG7) gene, the EHP Glyceraldehyde-3-phosphate dehydrogenase type 1 (Gly3PD) gene, the EHP ADP-ATP carrier protein gene, the EHP ADP-ATP carrier protein 4 gene, the EHP Glucose-6-phosphate dehydrogenase (ZWF1) gene and the EHP plasma membrane transporter gene.

According to some embodiments of the present invention the RNA molecule comprising at least one sequence capable of binding through complementary base pairing to an EHP target mRNA molecule or comprising at least one sequence at least partially complementary to an EHP target mRNA molecule comprises a nucleic acid sequence complementary to a nucleic acid sequence selected from the group consisting of SEQ ID NOs. 1-8.

According to some embodiments of the present invention the RNA molecule comprising at least one sequence having at least 90% sequence identity to an EHP target mRNA molecule or at least one sequence identical to at least 21 contiguous bases of an EHP target mRNA molecule comprises:
(a) a nucleic acid sequence identical to at least 21 contiguous bases of a nucleic acid sequence selected from the group consisting of SEQ ID NOs: 5-8, or
(b) a nucleic acid sequence complementary to a nucleic acid sequence selected from the group consisting of SEQ ID NO: 5-8.

According to some embodiments of the present invention the composition comprises at least one additional RNA sequence capable of directing cleavage of an EHP target mRNA molecule.

According to some embodiments of the present invention the chitosan is 75-100% deacetylated low molecular weight chitosan and the chitosan:RNA mass ratio (w/w) of the particle is in the range of 0.3-0.9.

According to some embodiments of the present invention the chitosan:RNA ratio is in the range of 0.45-0.55 and the molecular weight of the chitosan is in the range of 20-30 kDa.

According to some embodiments of the present invention the RNA content (w/w) of the nutraceutical composition is in the range of 0.01-10%.

According to some embodiments of the present invention the RNA content (w/w) of composition of the nutraceutical composition is in the range of 0.1%-5%.

According to some embodiments of the present invention ingestion of the composition or nutraceutical composition by the farmed crustacean results in increased survival, yield, growth rate, vigor, biomass, feed conversion, size, quality of taste and odor or stress tolerance of the farmed crustacean, compared to the same farmed crustacean ingesting feed devoid of RNA targeted to an EHP gene product.

According to some embodiments of the present invention the farmed crustaceans are selected from the group consisting of Shrimp, Prawns, Crabs, Lobsters and Crayfishes.

According to some embodiments of the present invention the farmed crustaceans are shrimp or prawns.

According to some embodiments of the present invention feeding is performed 2 to 5 times per day.

According to some embodiments of the present invention feeding is performed 5 times per day.

According to some embodiments of the present invention, shrimp with body weight between 3 to 30 milligrams are provided feed equaling 90-400% of the shrimp biomass per day, shrimp with body weight between 30 milligrams to 3 grams are provided feed equaling 7-90% of the shrimp biomass per day and shrimp with body weight between 3 to 7 grams or greater are provided feed equaling 4-7% of their biomass per day.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a diagram depicting the design of the EHP challenge. The large squares each represent a 500-L tank, filled with 200-L of artificial seawater. Each tank contains replicates of the four treatments (Treatments1-4: Treatment 1—Shrimps fed feed A containing chitosan-encapsulated dsRNA targeting the PTP2 gene; Treatment 2—Shrimps fed feed B containing a mix of chitosan-encapsulated dsRNAs targeting each of the four EHP target genes (PTP2, EhSWP1, ABCG7 and Gly3PD); Treatment 3—Shrimps injected with a cocktail of dsRNAs targeting each of the four EHP target genes (PTP2, EhSWP1, ABCG7 and Gly3PD), at a concentration of 1 μg of each dsRNA per gram shrimp; Treatment 4—positive control—shrimp fed feed without the addition of dsRNA and without injected dsRNA). The smaller rectangles represent baskets containing 15 shrimp each;

FIGS. 2A and 2B are histograms of EHP microsporidian loads in shrimp subjected to different treatments, measured 7 (FIG. 2A) and 12 (FIG. 2B) days following co-habitation with infected shrimp (day 0). Feed A=Treatment 1; Feed B=Treatment 2; dsRNA B=Treatment 3 and Positive Control=Treatment 4 (see above). Note the dramatic reduction in EHP load in shrimp fed with (Feed B), as well as injected with (dsRNA-B) the cocktail of dsRNA targeting 4 EHP genes. The different superscript letters represent statistically significant differences in the results;

FIG. 3 is a histogram of EHP microsporidian loads in shrimp vaccinated with dsRNA cocktails 1 (PTP2, SWP1, GLY3PD, ABCG7, SWP3, SWP7), 2 (SWP3, SWP7, OQS54254, Endochitinase, GLY3PD, ABCG7), 3 (PTP2, SWP1, SWP3, SWP7, OQS54254, Endochitinase) or 4 (PTP2, SWP1, OQS54254, Endochitinase, GLY3PD, ABCG7), and unvaccinated negative controls, 15 days after EHP infection.

### DESCRIPTION OF SPECIFIC EMBODIMENTS OF THE INVENTION

The present invention, in some embodiments thereof, relates to the use of particle-complexed RNAi for increasing resistance of farmed aquatic animals to microsporidian parasites.

In particular, the present invention provides modified chitin-RNA particles and methods for enhancing resistance to infection by Enterocytozooan pathogens of farmed aquatic crustaceans, such as shrimp and prawns.

The principles and operation of the present invention may be better understood with reference to the drawings and accompanying descriptions.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

Aquaculture, or farming or culturing of aquatic species, is an age-old but also fast-growing industry, of particular importance recently in light of dwindling numbers of wild fresh and salt water stocks, and the increased prominence of fish and seafood in the Western diet. Commonly cultured aquatic species now include fish, crustaceans, mollusks and even seaweed and echinoderms (sea cucumbers).

Along with the advantages of farming, high-density culture of aquatic populations, and the limited genetic variability of the farmed populations have created an opportunity for pathogenic organisms, and particularly viral and parasitic pathogens, to flourish and sometimes reach epidemic proportions, resulting in loss of income and significant reduction in stock populations.

While gene silencing by RNAi may be an attractive solution to the challenges of providing therapeutic treatment in an aquaculture environment (e.g. dsRNA can be amplified within the host organism), development of effective and cost-effective RNAi agents for enhancing resistance to intracellular parasites in cultured aquatic species has lagged.

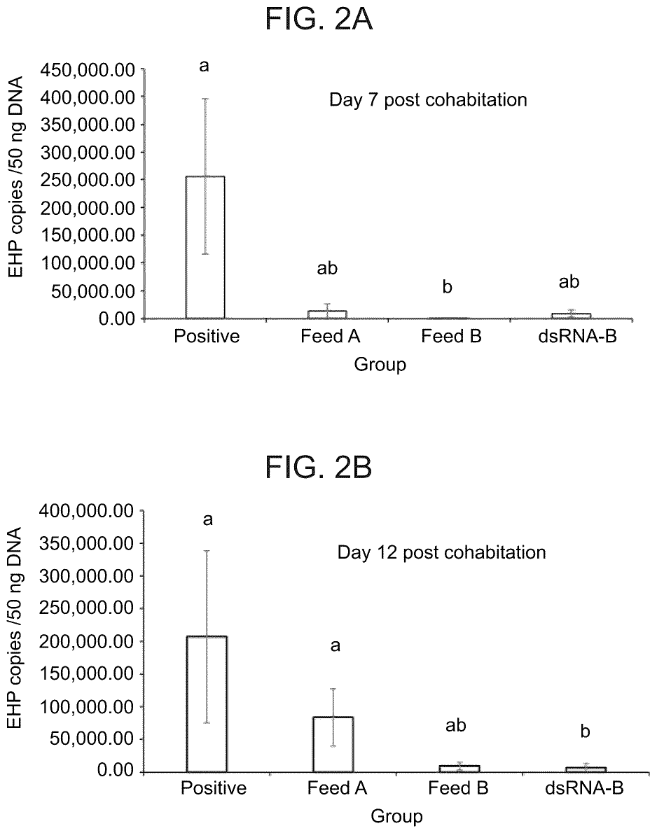

Whilst reducing the present invention to practice, the present inventors have developed chitosan-RNA particles comprising double stranded RNA (dsRNA) and/or single stranded RNA (ssRNA) targeted to microsporidian genes which, when ingested by or administered to farmed aquatic shrimp, effectively reduce microsporidian parasitic load in experimentally infected shrimp. Specifically, the instant inventors have uncovered that feeding a cocktail of chitosan-RNA particles targeting a number of microsporidian genes reduces the severity of Enterocytozoon hepatopenaei infection in farmed shrimp as effectively as direct injection of the silencing RNAs (see Example I and FIGS. 2A-B).

Thus, according to some embodiments of an aspect of the invention there is provided a composition comprising chitosan and at least one type of RNA molecule comprising a nucleic acid sequence at least 50 bases in length, wherein said RNA molecule is capable of silencing expression of a gene of a microsporidian parasitic in a farmed aquatic animal when administered to said farmed aquatic animal.

The term “chitin”, as used herein refers to a long-chain biopolymer of N-acetylglucosamine [i.e. 2-(acetylamino)-2-deoxy-D-glucose] in beta-1,4 linkage [poly (N-acetyl-1,4-beta-D-glucopyranosamine)]. Chitin is ubiquitous in insect exoskeletons and fungi and bacterial cell walls, and abundantly available commercially (see, for example, Sigma-Aldrich product No. C7170, Sigma-Aldrich, St Louis, MO). A distinction is made between alpha, beta and gamma chitin, which differ in the arrangement of their polymer chains (and, subsequently, in their mechanical properties): alpha chitin has alternating antiparallel arrangement of the chains (most common in crustaceans); beta chitin has a parallel arrangement of the polysaccharide chains (common in squids) and gamma chitin has two parallel chains statistically alternating with an antiparallel chain (common in fungi). The term “chitin” is synonymous with any one of alpha, beta or gamma chitin, unless otherwise indicated. Chitin can be natural chitin, from naturally chitin-containing organisms, recombinant chitin from organisms genetically engineered to produce chitin or synthetic, chemically synthesized chitin from polymerization of mixtures of glucosamine monomers or oligomers. Different types of chitin polymer arrangements are distinguishable, for example, by proton nuclear magnetic resonance (1H NMR) spectroscopy and matrix-assisted laser desorption ionization time-of-flight mass spectrometry (MALDI-TOF MS).

Chitin is fairly insoluble, dissolving only in strong solvent systems.

As used herein, the term “chitosan” refers to a deacetylated chitin polymer, which, in contrast to chitin, has free amino groups. Chitosan can be obtained by alkaline deacetylation of chitin, producing a chitin polymer with randomly acetylated amine groups, with a variety of properties depending on the degree of deacetylation and chain length. Chitosan is safe, non-toxic, easily dissolved in weak acidic solutions and can interact with polyanions to form complexes and gels. Chitosan can be characterized, inter alia, by the ratio of acetylated to non-acetylated (deacetylated) amine groups in the polymer, and by the average chain length of the polymers. Commercially available chitosan is usually provided as a powder, in alpha, beta or gamma orientation (according to the source of the chitin), with the degree of deacetylation (% DD) between 60 and 100%, and a molecular weight between about 4 to greater than 250 kDa, and in various degrees of purity. As used herein, the term “partially deacetylated chitosan” refers to chitosan which has been treated to acetylate a portion of the deacetylated, free amino groups. As in commercially available chitosan, partially deacetylated chitosan is characterized by the degree of deacetylation (% DD). The degree of deacetylation of chitosan can be determined by a variety of methods, usually classified into three categories: (1) spectroscopy (IR, (1)H NMR, (13)C NMR, (15)N NMR, and UV); (2) conventional (various types of titration, conductometry, potentiometry, ninhydrin assay, adsorption of free amino groups of chitosan by pictric acid) and (3) destructive (elemental analysis, acid or enzymatic hydrolysis of chitin/chitosan and followed by the DA measurement by colorimetry or high performance liquid chromatography, pyrolysis-gas chromatography, and thermal analysis using differential scanning calorimetry) methods.

According to some aspects of the invention, the chitosan-RNA particle comprises untreated chitosan, i.e. chitosan which has not been treated to reduce the degree of deacetylation. It will be appreciated that the degree of deacetylation of a chitosan preparation relates to the distribution of chitosan molecules within the population of chitosan being used for the methods and compositions of the invention. Thus, the chitosan of the chitosan-RNA particle of the invention can be characterized as being deacetylated within a range of values for degree of deacetylation. In particular embodiments, the particulate composition comprises chitosan having a degree of deacetylation in the range of 65-100%, in the range of 70-100%, in the range of 75-100%, in the range of 80-100%, in the range of 85-100%, 90-100%, 95-100%, in the range of 65-95%, 71-91%, 75-89% or 78-85%. In other embodiments the particulate composition comprises chitosan having a degree of deacetylation of 65%, 67%, 70%, 72%, 74%, 76%, 78%, 80%, 82%, 84%, 86%, 88%, 90%, 91%, 92%, 93%, 94%, 95%, 96%, 97%, 98%, 99% or 100%. In specific embodiments of the invention the particulate composition comprises chitosan having a degree of deacetylation of 75-100%. In other embodiments, the particulate composition comprises chitosan having a degree of deacetylation of 80-95%. In further embodiments, the particulate composition comprises chitosan having a degree of deacetylation of 90%.

The chitosan-RNA particle may be formulated using a chitosan:RNA ratio ranging from high (8, 10, 20, 40 or greater) to low (<1.0).

In some embodiments, the RNAi of the chitosan-RNA particle comprising chitosan-RNAi particles having a low (<1.0) chitosan:RNA mass ratio. Thus, in some embodiments of some aspects of the invention, the chitosan-RNA particle comprises particles having a chitosan:RNA ratio (w/w) in the range of 0.2-0.95. In some embodiments, the chitosan:RNA ratio is in the range of 0.25-0.9, 0.3-0.85, 0.35-0.80, 0.4-0.9, 0.3-0.8, 0.2-0.75 and 0.25-0.5. In other embodiments, the chitosan:RNA ratio of the particles is about 0.2, 0.25, 0.30, 0.35, 0.40, 0.45, 0.50, 0.55, 0.60, 0.65, 0.70, 0.75, 0.80, 0.85, 0.90, 0.95. In specific embodiments, the particulate composition comprises particles having a chitosan:RNA ratio (w/w) of 0.45-0.55, and, in particular, chitosan:RNA ratio (w/w) of 0.5. In other specific embodiments, the chitosan is 75-100% deacetylated, low molecular weight chitosan, and the chitosan:RNA mass ratio (w/w) of the particle is in the range of 0.3-0.9. In further embodiments, the chitosan is 80-95% deacetylated, low molecular weight chitosan, and the chitosan:RNA mass ratio (w/w) of the particle is in the range of 0.45-0.55.

In some embodiments, the molecular weight of the chitosan in the chitosan-RNA particle is in the range of 5-100 kDa. In other embodiments, the molecular weight of the chitosan in the particle is in the range of 10-85 kDa, in the range of 12-90 kDa, in the range of 15-75 kDa, 17-65 kDa, 20-60 kDa, 25-55 kDa, 20-50 kDa, 20-40 kDa, 23-38 kDa, 25-35 kDa, or 20-30 kDa. In still other embodiments, the chitosan is a low molecular weight chitosan, having a degree of deacetylation of about 10, 12, 14, 16, 18, 20, 22, 24, 26, 28, 30, 32, 34, 36, 38, 40, 42, 44, 46, 48, 50, 52, 55, 58, 60, 62, 65, 68, 70, 72, 75, 78, 80, 82, 85, 88, 90, 92, 95, 98, 99 or 100 kDa. In particular embodiments the molecular weight of the chitosan in the particle is in the range of 10-50 kDa, 20-30 kDa or 30 kDa.

In specific embodiments, the chitosan-RNA particle of the invention comprises chitosan:RNA particles comprising 50-100% deacetylated chitosan, and having a chitosan:RNA ratio in the range of 0.45-0.55, or 0.5 and chitosan of a molecular weight in the range of 20-30 kDa, in some embodiments 30 kDa.

According to some aspects of the invention, the chitosan-RNA particles are particles, i.e. having a particle size within the micrometer (μm) rather than within the nanometer (nm) range.

Particle size may be measured by z-average diameter, for example, using the Mastersizer (Malvernpanalytical, Ltd), or using microscopic techniques with microscopic resolution above 1 micron.

In some embodiments, the composition of the invention comprises greater than 50% of the particles in the range of 10-300 μm. In other embodiments, the chitosan-RNA particles of the composition have mean particle size of about 10, about 15, about 18, about 20, about 27, about 30, about 32, about 35, about 38, about 40, about 43, about 45, about 47, about 50, about 55, about 60, about 65, about 70, about 75, about 80, about 85, about 90, about 95, about 100, about 110, about 120, about 130, about 140, about 150, about 160, about 170, about 180, about 190, about 200, about 210, about 220, about 230, about 240, about 250, about 260, about 270, about 280, about 290 or about 300 μm.

According to some embodiments of the invention the chitosan-RNA particle comprises at least one double stranded RNA (dsRNA). As used herein the term “dsRNA” relates to two strands of anti-parallel polyribonucleic acids held together by base pairing. “dsRNA” may also be described as an “dsRNA duplex” or “RNA duplex” or “duplex RNA”. The two strands can be of identical length or of different lengths provided there is enough sequence homology between the two strands that a double stranded structure is formed with at least 80%, 90%, 95% or 100% complementarity over the entire length. The level of complementation between the strands of dsRNAs suitable for use with the invention as described herein is selected to provide at least sufficient complementation to affect gene silencing of the target RNA molecules of interest.

In other specific embodiments of the invention, the chitosan-RNA particle comprises at least one single stranded RNA (ssRNA). As used herein the term “ssRNA” relates to a single strand of polyribonucleic acid. When the single stranded RNA is a silencing RNA directed to a specific target sequence in a target organism (e.g. EHP sequence such as PTP2, EhSWP1, ABCG7 or Gly3PD, etc), the ssRNA can be either identical to the target RNA sequence or a portion thereof (“Sense ssRNA”), or, alternatively, complementary to the target RNA sequence or a portion thereof (“Anti-sense ssRNA”). In some embodiments, the chitosan-RNA particles can comprise a combination of sense- and antisense ssRNA.

It will be noted that some single stranded RNA sequences may comprise self-complementary portions (e.g. “pallindromic” or “sub-sequences”) which, under certain conditions, can form segments of internal base pairing, resulting in partial double stranded RNA formation from the single-stranded RNA of the particles of the invention. It will be noted that the ss or dsRNA can be defined in terms of the nucleic acid sequence of the DNA encoding the target gene transcript, and it is understood that an antisense ss or dsRNA sequence corresponding to the coding sequence of a gene comprises an RNA complement of the gene's coding sequence, or other sequence of the gene which is transcribed into RNA.

Ss or dsRNA can be used for downregulation of gene expression by RNA silencing. As used herein, the phrase “RNA silencing” refers to a group of regulatory mechanisms [e.g. RNA interference (RNAi), transcriptional gene silencing (TGS), post-transcriptional gene silencing (PTGS), quelling, co-suppression, and translational repression] mediated by RNA molecules which result in the inhibition or “silencing” of the expression of a corresponding protein-coding gene, or, in some cases, gene encoding a functional RNA product. RNA silencing has been observed in many types of organisms, including plants, animals (vertebrates and invertebrates), and fungi.

The RNA can be a ss or dsRNA which is capable of specifically inhibiting or “silencing” the expression of a target gene. In some embodiments the RNA is capable of preventing complete processing (e.g. the full translation and/or expression) of an mRNA molecule through a post-transcriptional silencing mechanism. When used for gene silencing, RNA include noncoding RNA molecules, for example RNA duplexes comprising paired strands, as well as precursor RNAs from which such small non-coding RNAs can be generated. Exemplary RNA that can be used for RNA silencing include siRNA duplexes, miRNAs and shRNAs. In one embodiment, the RNA is capable of inducing RNA interference. In another embodiment, the RNA is capable of mediating translational repression. In yet another embodiment, the RNA is capable of directing cleavage of a target RNA. Cleavage of such a target RNA can be effected via an RNAi pathway, as described in detail hereinbelow.

The inhibitory RNA sequence of the RNA can be greater than 90% identical, or even 100% identical, to the portion of the target gene transcript. According to an embodiment of the invention, the RNA silencing agent is specific to the target RNA (e.g., E. hepatopenaei PTP2, EhSWP1, ABCG7, Gly3PD, SWP3, SWP7, Endochitinase or New Spore Wall Protein OQS54254) and does not cross-inhibit or silence a gene or a splice variant which exhibits 99% or less global homology to the target gene, e.g., less than 98%, 97%, 96%, 95%, 94%, 93%, 92%, 91%, 90%, 89%, 88%, 87%, 86%, 85%, 84%, 83%, 82%, 81% global homology to the target gene.

In some embodiments, the RNA, for example, the duplex region of the dsRNA may be defined functionally as a nucleotide sequence that is capable of binding through complementary base pairing to a target mRNA molecule of the microsporidian parasite—e.g. hybridizing with a portion of the target gene transcript under stringent conditions (e.g., 400 mM NaCl, 40 mM PIPES pH 6.4, 1 mM EDTA, 60 degrees C. hybridization for 12-16 hours; followed by washing).

In some embodiments, the inhibitory RNA sequence of the chitosan-RNA particle is a single stranded, sense RNA sequence, comprising at least one sequence having at least 90% sequence identity to the target gene transcript (e.g. target mRNA molecule).

The length of the double-stranded or single stranded nucleotide sequences complementary or identical to the target gene transcript may be at least about 18, 19, 20, 21, 25, 40, 50, 75, 100, 150, 200, 250, 300, 400, 491, 500, 600 or at least 750 or more bases. In some embodiments, the double-stranded or single stranded nucleotide sequences complementary or identical to the target gene transcript comprise at least 20, at least about 21, 25, 40, 50, 75, 100, 125, 150, 200, 250, 300, 400, 491, 500, 600 or at least 750 or more contiguous bases. In some embodiments, the length of the targeted mRNA sequence can be at least 20, at least 40, at least 50, at least 75, at least 100, at least 150, at least 200, at least 250, at least 400, at least 500, at least 600, at least 750 bases in length. It will be appreciated that each of the lengths represents a separate embodiment.

In some embodiments of some aspects of the invention, the length of the single stranded or double-stranded nucleotide sequence is approximately from about 40 to about 450, about 50 to about 450, about 60 to about 350, about 60 to about 300 and about 80 to about 250 nucleotides in length for genes of microsporidian parasites of farmed aquatic animals thereof. In some embodiments of some aspects of the invention, the length of the single or double-stranded nucleotide sequence is approximately from 50 to 350 nucleotides in length for genes of microsporidian parasites of farmed aquatic animals thereof. In specific embodiments, the length of the single or double-stranded nucleotide sequence is 125 or 250. It will be appreciated that each individual range represents a distinct and separate embodiment.

The term “corresponds to” as used herein means a polynucleotide sequence possessing homology (or identity, for example, 100% homology) to all or a portion of a reference polynucleotide sequence. In contradistinction, the term “complementary to” is used herein to mean that the complementary sequence is homologous (or identical, i.e. 100% homologous) to all or a portion of the complement of a reference polynucleotide sequence. For example, the nucleotide sequence “TATAC” corresponds to a reference sequence “TATAC” and is complementary to a reference sequence “GTATA”.

The present teachings relate to various lengths of ss or dsRNA, whereby the shorter version (shorter or equal to 50 bp (e.g., 17-50)), is referred to as siRNA or miRNA. Longer ss or dsRNA molecules of 51-600 are referred to herein as ss or dsRNA, which can be further processed to siRNA molecules. According to some embodiments, the nucleic acid sequence of the ss dsRNA is greater than 15 bases or base pairs in length. According to yet other embodiments, the nucleic acid sequence of the ss or dsRNA is 19-25 bases or base pairs in length, 30-100 bases or base pairs in length, 100-250 bases or base pairs in length or 100-500 bases or base pairs in length. According to still other embodiments, the ss or dsRNA is 300-600 bases or base pairs in length, 350-500 bases or base pairs in length or 400-450 bases or base pairs in length. In some embodiments, the ss or dsRNA is 400 bases or base pairs in length. It will be appreciated that each individual range of RNA lengths represents a separate and distinct embodiment.

The use of long ss or dsRNAs (i.e. RNA greater than 50 bp) has been very limited owing to the belief that these longer regions of RNA will result in the induction of non specific response. However, the use of long RNAs can provide numerous advantages in that the cell can select the optimal silencing sequence alleviating the need to test numerous siRNAs; long ss or dsRNAs will allow for silencing libraries to have less complexity than would be necessary for siRNAs; and, perhaps most importantly, long ss or dsRNA could prevent escape mutations when used as therapeutics.

Various studies demonstrate that long RNAs can be used to silence gene expression without inducing the stress response or causing significant off-target effects—see for example [Strat et al., Nucleic Acids Research, 2006, Vol. 34, No. 13 3803-3810; Bhargava A et al. Brain Res. Protoc. 2004; 13:115-125; Diallo M., et al., Oligonucleotides. 2003; 13:381-392; Paddison P. J., et al., Proc. Natl Acad. Sci. USA. 2002; 99:1443-1448; Tran N., et al., FEBS Lett. 2004; 573:127-1341.

In some embodiments, the dsRNA can comprise siRNA duplexes. The term “siRNA” refers to small inhibitory RNA duplexes (generally between 17-30 basepairs, but also longer e.g., 31-50 bp) that induce the RNA interference (RNAi) pathway. Typically, siRNAs are chemically synthesized as 21mers with a central 19 bp duplex region and symmetric 2-base 3′-overhangs on the termini, although it has been recently described that chemically synthesized RNA duplexes of 25-30 base length can have as much as a 100-fold increase in potency compared with 21mers at the same location. The observed increased potency obtained using longer RNAs in triggering RNAi is theorized to result from providing Dicer with a substrate (27mer) instead of a product (21mer) and that this improves the rate or efficiency of entry of the siRNA duplex into RISC.

It has been found that position of the 3′-overhang influences potency of an siRNA and asymmetric duplexes having a 3′-overhang on the antisense strand are generally more potent than those with the 3′-overhang on the sense strand (Rose et al., 2005). This can be attributed to asymmetrical strand loading into RISC, as the opposite efficacy patterns are observed when targeting the antisense transcript.

The strands of a double-stranded interfering RNA (e.g., an siRNA) may be connected to form a hairpin or stem-loop structure (e.g., an shRNA). Thus, as mentioned the dsRNA of some embodiments of the invention may also be a short hairpin RNA (shRNA). A short hairpin RNA (shRNA) can also result from internal stem- and loop base-pairing within a single stranded RNA of the invention.

The term “shRNA”, as used herein, refers to an RNA agent having a stem-loop structure, comprising a first and second region of complementary sequence, the degree of complementarity and orientation of the regions being sufficient such that base pairing occurs between the regions, the first and second regions being joined by a loop region, the loop resulting from a lack of base pairing between nucleotides (or nucleotide analogs) within the loop region. The number of nucleotides in the loop is a number between and including 3 to 23, or 5 to 15, or 7 to 13, or 4 to 9, or 9 to 11. It will be appreciated that each of the ranges noted here represents a separate embodiment of the invention. Some of the nucleotides in the loop can be involved in base-pair interactions with other nucleotides in the loop. Examples of oligonucleotide sequences that can be used to form the loop include 5′-UUCAAGAGA-3′ (Brummelkamp, T. R. et al. (2002) Science 296: 550) and 5′-UUUGUGUAG-3′ (Castanotto, D. et al. (2002) RNA 8:1454). It will be recognized by one of skill in the art that the resulting single chain oligonucleotide forms a stem-loop or hairpin structure comprising a double-stranded region capable of interacting with the RNAi machinery.

According to the present teachings, the ss or dsRNA molecules may be naturally occurring or synthetic.

Synthesis of RNA suitable for use with some embodiments of the invention can be affected as follows. First, the target mRNA (e.g. mRNA of a microsporidian parasite pathogenic to farmed aquatic animals, e.g. crustaceans) sequence is scanned downstream of the AUG start codon for AA dinucleotide sequences. Occurrence of each AA and the 3′ adjacent 19 nucleotides is recorded as potential siRNA target sites. Preferably, siRNA target sites are selected from the open reading frame, as untranslated regions (UTRs) are richer in regulatory protein binding sites. UTR-binding proteins and/or translation initiation complexes may interfere with binding of the siRNA endonuclease complex [Tuschl Chem Biochem. 2:239-245]. It will be appreciated though, that siRNAs directed at untranslated regions may also be effective, as demonstrated for GAPDH wherein siRNA directed at the 5′ UTR mediated about 90% decrease in cellular GAPDH mRNA and completely abolished protein level (see Ambion website—techlib 91/912).

Second, potential target sites are compared to an appropriate genomic database (e.g., farmed aquatic animals, crustaceans) using any sequence alignment software, such as the BLAST software available from the NCBI server (www(dot)ncbi(dot)nlm(dot)nih(dot)gov/BLAST/). Putative target sites which exhibit significant homology to other coding sequences are identified and filtered out.

Thus, in particular embodiments, the RNA molecule capable of silencing gene expression of a gene of the parasitic microsporidian is at least one RNA partially complementary to the microsporidian nucleic acid target molecule, or binds to the target molecule through complementary base pairing, or has at least one sequence identical to at least 20 contiguous bases of the target microsporidian mRNA molecule. In particular embodiments, the at least one RNA which is at least partially complementary to the microsporidian target nucleic acid molecule, or binds to the microsporidian target molecule through complementary base pairing, or has at least one sequence identical to at least 20 contiguous bases of the microsporidian target mRNA molecule does not have any significant homology to any of the farmed aquatic animal host gene sequences.

Qualifying target sequences are selected as template for siRNA synthesis. Preferred sequences are those including low G/C content as these have proven to be more effective in mediating gene silencing as compared to those with G/C content higher than 55%. Several target sites are preferably selected along the length of the target gene for evaluation. For better evaluation of the selected siRNAs, a negative control is preferably used in conjunction. Negative control siRNA can include the same nucleotide composition as the siRNAs but lack significant homology to the genome. Thus, a scrambled nucleotide sequence of the siRNA is often used, provided it does not display any significant homology to any other gene.

The ss or dsRNA may be synthesized using any method known in the art, including either enzymatic syntheses or solid-phase syntheses. These are especially useful in the case of short polynucleotide sequences with or without modifications as explained above. Equipment and reagents for executing solid-phase synthesis are commercially available from, for example, Applied Biosystems. Any other means for such synthesis may also be employed; the actual synthesis of the oligonucleotides is well within the capabilities of one skilled in the art and can be accomplished via established methodologies as detailed in, for example: Sambrook, J. and Russell, D. W. (2001), “Molecular Cloning: A Laboratory Manual”; Ausubel, R. M. et al., eds. (1994, 1989), “Current Protocols in Molecular Biology,” Volumes I-III, John Wiley & Sons, Baltimore, Maryland; Perbal, B. (1988), “A Practical Guide to Molecular Cloning,” John Wiley & Sons, New York; and Gait, M. J., ed. (1984), “Oligonucleotide Synthesis”; utilizing solid-phase chemistry, e.g. cyanoethyl phosphoramidite followed by deprotection, desalting, and purification by, for example, an automated trityl-on method or HPLC.

The ss or dsRNA can be a mixture of long and short dsRNA molecules such as, dsRNA, siRNA, siRNA+dsRNA, siRNA+miRNA, hpRNA or a combination of same. According to a specific embodiment, the dsRNA is an siRNA (100%). The chitosan-RNA particles can also comprise a mixture of sense and anti-sense single stranded RNA.

It will be appreciated that the ss or dsRNA of some embodiments of the invention need not be limited to those molecules containing only RNA, but further encompasses chemically-modified nucleotides and non-nucleotides.

In some embodiments, the RNA provided herein can be functionally associated with a cell-penetrating peptide. As used herein, a “cell-penetrating peptide” is a peptide that comprises a short (about 12-30 residues) amino acid sequence or functional motif that confers the energy-independent (i.e., non-endocytotic) translocation properties associated with transport of the membrane-permeable complex across the plasma and/or nuclear membranes of a cell. The cell-penetrating peptide used in the membrane-permeable complex of some embodiments of the invention preferably comprises at least one non-functional cysteine residue, which is either free or derivatized to form a disulfide link with a double-stranded ribonucleic acid that has been modified for such linkage. Representative amino acid motifs conferring such properties are listed in U.S. Pat. No. 6,348,185, the contents of which are expressly incorporated herein by reference. The cell-penetrating peptides of some embodiments of the invention preferably include, but are not limited to, penetratin, transportan, pIsl, TAT(48-60), pVEC, MTS, and MAP. On the other hand, in some embodiments, the use of a cell penetrating peptide or transformation agent is undesirable-thus, in some embodiments, the ss or dsRNA is provided lacking, devoid of a cell penetrating peptide or any other form of cell penetrating agents, transformation enhancing agents. It will be noted that, in the context of the present invention, cell penetrating peptide, cell penetrating agents and transformation enhancing agents do not include chitosan or a polymer such as polyethylene glycol (PEG).

According to another embodiment the ss or dsRNA may be a microRNA precursor, or “pri-miRNA”.

The term “microRNA”, “miRNA”, and “miR” are synonymous and refer to a collection of non-coding single-stranded RNA molecules of about 19-28 nucleotides in length, which regulate gene expression. miRNAs are found in a wide range of organisms (viruses.fwdarw.humans) and have been shown to play a role in development, homeostasis, and disease etiology.

Below is a brief description of the mechanism of miRNA activity.

Genes coding for miRNAs are transcribed leading to production of an miRNA precursor “the pri-miRNA”. The pri-miRNA is typically part of a polycistronic RNA comprising multiple pri-miRNAs. The pri-miRNA may form a hairpin with a stem and loop. The stem may comprise mismatched bases.

The hairpin structure of the pri-miRNA is recognized by Drosha, which is an RNase III endonuclease. Drosha typically recognizes terminal loops in the pri-miRNA and cleaves approximately two helical turns into the stem to produce a 60-70 nucleotide precursor known as the pre-miRNA. Drosha cleaves the pri-miRNA with a staggered cut typical of RNase III endonucleases yielding a pre-miRNA stem loop with a 5′ phosphate and ~2 nucleotide 3′ overhang. It is estimated that approximately one helical turn of stem (~10 nucleotides) extending beyond the Drosha cleavage site is essential for efficient processing. The pre-miRNA is then actively transported from the nucleus to the cytoplasm by Ran-GTP and the export receptor Ex-portin-5.

The double-stranded stem of the pre-miRNA is then recognized by Dicer, which is also an RNase III endonuclease. Dicer may also recognize the 5′ phosphate and 3′ overhang at the base of the stem loop. Dicer then cleaves off the terminal loop two helical turns away from the base of the stem loop leaving an additional 5′ phosphate and ~2 nucleotide 3′ overhang. The resulting siRNA-like duplex, which may comprise mismatches, comprises the mature miRNA and a similar-sized fragment known as the miRNA*. The miRNA and miRNA* may be derived from opposing arms of the pri-miRNA and pre-miRNA. MiRNA* sequences may be found in libraries of cloned miRNAs but typically at lower frequency than the miRNAs.

Although initially present as a double-stranded species with miRNA*, the miRNA eventually become incorporated as a single-stranded RNA into a ribonucleoprotein complex known as the RNA-induced silencing complex (RISC). Various proteins can form the RISC, which can lead to variability in specificity for miRNA/miRNA* duplexes, binding site of the target gene, activity of miRNA (repress or activate), and which strand of the miRNA/miRNA* duplex is loaded in to the RISC.

When the miRNA strand of the miRNA:miRNA* duplex is loaded into the RISC, the miRNA* is removed and degraded. The strand of the miRNA:miRNA* duplex that is loaded into the RISC is the strand whose 5′ end is less tightly paired. In cases where both ends of the miRNA:miRNA* have roughly equivalent 5′ pairing, both miRNA and miRNA* may have gene silencing activity.

The RISC identifies target nucleic acids based on high levels of complementarity between the miRNA and the mRNA, especially by nucleotides 2-7 of the miRNA.

A number of studies have looked at the base-pairing requirement between miRNA and its mRNA target for achieving efficient inhibition of translation (reviewed by Bartel 2004, Cell 116-281). In mammalian cells, the first 8 nucleotides of the miRNA may be important (Doench & Sharp 2004 GenesDev 2004-504). However, other parts of the microRNA may also participate in mRNA binding. Moreover, sufficient base pairing at the 3′ can compensate for insufficient pairing at the 5′ (Brennecke et al, 2005 PLoS 3-e85). Computation studies, analyzing miRNA binding on whole genomes have suggested a specific role for bases 2-7 at the 5′ of the miRNA in target binding but the role of the first nucleotide, found usually to be “A” was also recognized (Lewis et at 2005 Cell 120-15). Similarly, nucleotides 1-7 or 2-8 were used to identify and validate targets by Krek et al (2005, Nat Genet 37-495).

The target sites in the mRNA may be in the 5′ UTR, the 3′ UTR or in the coding region. Interestingly, multiple miRNAs may regulate the same mRNA target by recognizing the same or multiple sites. The presence of multiple miRNA binding sites in most genetically identified targets may indicate that the cooperative action of multiple RISCs provides the most efficient translational inhibition.

MiRNAs may direct the RISC to downregulate gene expression by either of two mechanisms: mRNA cleavage or translational repression. The miRNA may specify cleavage off the mRNA if the mRNA has a certain degree of complementarity to the miRNA. When a miRNA guides cleavage, the cut is typically between the nucleotides pairing to residues 10 and 11 of the miRNA. Alternatively, the miRNA may repress translation if the miRNA does not have the requisite degree of complementarity to the miRNA. Translational repression may be more prevalent in animals since animals may have a lower degree of complementarity between the miRNA and binding site.

It should be noted that there may be variability in the 5′ and 3′ ends of any pair of miRNA and miRNA*. This variability may be due to variability in the enzymatic processing of Drosha and Dicer with respect to the site of cleavage. Variability at the 5′ and 3′ ends of miRNA and miRNA* may also be due to mismatches in the stem structures of the pri-miRNA and pre-miRNA. The mismatches of the stem strands may lead to a population of different hairpin structures. Variability in the stem structures may also lead to variability in the products of cleavage by Drosha and Dicer.

Thus, according to some embodiments, the chitosan-RNA particle comprises at least one RNA sequence capable of binding through complementary base pairing to a target mRNA molecule of the microsporidian pathogenic in farmed aquatic animals, and particularly, farmed aquatic crustaceans. The target mRNA can be a transcript of any gene sequences of the microsporidian parasite. Genes from the pathogenic microsporidian parasite having sequences complementary to such target mRNA sequences are considered target genes, provided that the target sequences lack substantial homology to the host gene.

In another embodiment, the chitosan-dsRNA particle comprises at least one RNA sequence at least partially complementary to a target mRNA of a microsporidian parasite pathogenic in farmed crustaceans, and particularly, farmed aquatic crustaceans.

In yet another embodiment, the chitosan-dsRNA particle comprises at least one RNA sequence at least 90% identical to a target mRNA of a microsporidian parasite pathogenic in farmed animals, and particularly, farmed aquatic crustaceans.

As used herein, the phrase “microsporidian pathogen target gene” or “microsporidian target gene” or “microsporidian parasite target gene” is defined as a microsporidian gene, the expression of which is critical, or essential to the toxicity, growth or pathogenicity of the microsporidian. The term “gene” is used broadly to refer to any segment of the microsporidian nucleic acid associated with a biological function. Thus, genes include coding sequences and/or the regulatory sequences required for their expression. For example, gene refers to a nucleic acid fragment that expresses mRNA or functional RNA, and optionally encodes a specific protein.

Microsporidian pathogen target genes include, but are not limited to structural genes for microsporidian proteins, genes for functional proteins (e.g. genes associated with replication, nutrition and energy metabolism) and other sequences associated with disease or disease symptoms in the host aquatic animal (e.g., farmed aquatic crustacean).

As used herein, the phrase “microsporidian pathogen gene product” refers to a product of the expression of a microsporidian pathogen gene—including, but not limited to the RNA transcript of the microsporidian (e.g. EHP) pathogen gene and a peptide or polypeptide encoded by a sequence of a microsporidian pathogen (e.g. EHP) gene.

Treating or preventing the microsporidian pathogen infection is achieved by reducing the expression of a microsporidian pathogen gene. Thus, in some embodiments, the at least one microsporidian parasitic pathogen gene is a microsporidian gene whose expression is initiated or increased during the course of the microsporidian pathogen infection. For example, Wiredu Boakye et al (Environmental Microbiol, 2017, doi:1111/1462-2920.13734) discloses microsporidian genes, particularly E. hepatopaenia genes, which are critical to the parasite's energy metabolism, the expression of which can be altered (e.g. decreased), during the microsporidian pathogen infection.

Tables 1A and 1B provides a non-limiting list of microsporidian genii and target tissues associated with diseases of farmed aquatic animals (e.g. fish, crustaceans), which comprise sequences which can be targets for reduction in expression by the chitosan-dsRNA particle of the invention.

TABLE I
Diseases and Pathogenic Microsporidian in Aquaculture
Table 1A The described genera of Microsporidia infecting fish

| Genus | Example host | Target organ/tissue | Taxonomic attribution |
|---|---|---|---|
| Amazonspora | Hassar orestis | Gill | Azevedo & Matos, 2003 |
| Dasyatispora | Dasyatis pastinaca | Musculature | Diamant et al., 2010 |
| Glugea | Gasterosteus aculeatus | Various | Thelohan, 1891 |
| Heterosporis | Pseudocrenilabrusmulticolor | Musculature | Schubert, 1969 |
| Ichthyosporidium | Crenilabrus melops | Musculature | Caullery and Mesnil, 1905 |
| Kabatana | Oncorhynchus tshawytscha | Musculature | Lom et al., 2000 |
| Loma | Oncorhynchus tshawytscha | Gill | Morrison and Sprague, 1981 |
| Microfilium | Lutjanus fulgens | Gill | Faye et al., 1991 |
| Microgemma | Chelon labrosus | Liver | Ralphs and Matthews, 1986 |
| Microsporidium | Prosopium williamsoni | Various | Balbiani, 1884 |
| Myosporidium | Merluccius capensis | Musculature | Baquero et al., 2005 |
| Neonosemoides | Tilapia zilli | Gill | Faye and Togubaye, 1996 |
| Nucleospora | Oncorhynchus tshawytscha | Kidney | Hedrick et al., 1991 |
| Ovipleistophora | Rutilus rutilus | Ovary | Pekkarinnen et al., 2002 |
| Paranucleospora | Salmo salar | Haematopoietic | Nylund et al., 2010 |
| Pleistophora | Mugil auratus | cells | Gurley, 1893 |
| Potaspora | Potamorhaphis guianensis | Various | Casal et al., 2008 |
| Pseudoloma | Danio rerio | Abdominal | Matthews et al., 2001 |
| Spraguea | Lophius piscatorius | lining (serosa) | Weissenberg, 1976 |
| Tetramicra | Scophthlamus maximus | Nervous | Matthews and Matthews, 1980 |

Table 1B: The described genera of Microsporidia infecting aquatic arthropod hosts

| Genus | Example host | Target organ/tissue | Taxonomic attribution |
|---|---|---|---|
| Abelspora | Carcinus maenas | Hepatopancreas | Azevedo, 1987 |
| Agglomerata | Sida crystallina | Not known | Larsson and Yan, 1988 |
| Agmasoma | Penaeus setiferus | Musculature | Hazard and Oldacre, 1975 |
| Alfvenia | Acanthocyclops vernalis | Adipose tissue | Larsson, 1983 |
| Ambylospora | Mesocyclops albicans | Not known | Hazard&Oldacre, 1975 |
| Ameson | Callinectes sapidus | Musculature | Sprague, 1977 |
| Baculea | Daphnia pulex | Not known | Loube\grave{ }s& Akbarieh, 1978 |
| Berwaldia | Daphnia pulex | Adipose tissue | Larsson, 1981 |
| Binucleata | Daphnia magna | Epithelial cells | Refardt et al. (2008) |
| Binucleospora | Candona spp. | Adipose tissue | Bronnvall, 1995 |
| Cougourdella | Megacyclops viridis | Adipose tissue | Hesse, 1935 |
| Cucumispora | Dikerogammarus villosus | Musculature | Ovcharenko et al., 2010 |
| Desomozoon | Lepeophteirus salmonis | Epithelial cells | Freeman et al., 2003 |
| Dictyocoela | Gammarus spp. | Gonadal tissues | Terry et al., 2004 |
| EncEHPalitozoon | Macrocylops distinctus | Not known | Levaditi et al., 1923 |
| Enterocytozoon | Penaeus monodon | Hepatopancreas | Desportes et al., 1985 |
| Enterospora | Cancer pagurus | Hepatopancreas | Stentiford et al., 2007 |
| Facilispora | Lepeophteirus spp. | Not known | Jones et al., 2012 |
| Fibrillanosema | Crangonyx pseudogracilis | Gonadal tissues | Galbreath et al., 2003 |
| Flabelliformab | Candona spp. | Not known | Canning et al., 1991 |
| Glugoides | Daphnia magna/pulex | Gut epithelium | Larsson et al., 1995 |
| Gurleya | Daphnia maxima | Hypodermal tissue | Doflein, 1898 |
| Gurleyides | Ceriodaphnia reticulata | Adipose tissue | Voronin, 1986 |
| Hamiltosporidium | Daphnia magna | Adipose tissue | Haag et al., 2011 |
| Hepatospora | Eriocheir sinensis | Hepatopancreas | Stentiford et al., 2011 |
| Holobispor | Thermocyclops oithonoides | Not known | Voronin, 1986 |
| Inodosporus | Palaemonetes spp. | Musculature | Overstreet and Weidner, 1974 |
| Lanatospora | Macrocyclops albidus | Adipose tissue | Voronin, 1986 |
| Larssonia | Daphnia pulex | Adipose tissue | Vidtmann and Sokolova, 1994 |
| Marssoniella | Cyclops strennus | Not known | Lemmermann, 1900 |
| Mrazekia | Asellus aquaticus | Adipose tissue | Le'ger and Hesse, 1916 |
| Myospora | MetanEHProps challengeri | Musculature | Stentiford et al., 2010 |
| Nadelspora | Cancer magister | Musculature | Olson et al., 1994 |
| Nelliemelba | Boeckella triarticulata | Musculature | Larsson, 1983 |
| Norlevinea | Daphnia longispina | Not known | Va'vra, 1984 |
| Nosema | Artemia salina | Musculature | Na\ddot{ }geli, 1857 |
| Octosporea | Daphnia magna | Musculature | Flu, 1911 |
| Ordospora | Daphnia magna | Gut epithelium | Larsson et al., 1997 |
| Ormieresia | Carcinus mediterraneus | Musculature | Vivare\grave{ }s et al., 1977 |
| Parathelohania | Microcyclops varicans | Systemic | Codreanu, 1966 |
| Paranucleospora | Lepeophteirus salmonis | Epithelial cells | Nylund et al., 2010 |
| Perezia | Litopenaeus setiferus | Musculature | Le'gerandDuboscq, 1909 |
| Pleistophora | Crangon spp. | Musculature | Gurley, 1893 |
| Pyrotheca | Cyclops albidus | Not known | Hesse, 1935 |
| Thelohania | Crangon vulgaris | Musculature | Henneguy, 1892 |
| Trichotuzetia | Cyclops vicinus | Systemic | Va'vra et al., 1997 |
| Triwangia | Caridina formosae | Connective tissues | Wang et al., 2013 |
| Tuzetia | Macrocyclops albidus | Adipose tissues | Maurand et al., 1971 |
| Vairimorpha | Cheraz destructor | Musculature | Pilley, 1976 |
| Vavraia | Crangon crangon | Musculature | Weiser, 1977 |

In one embodiment, the at least one microsporidian pathogen gene is selected from the group consisting of mitosome proteins, enzymes of microsporidian replication (e.g. microsporidian RNA polymerases, helicases), microsporidian proteases and microsporidian chitin-binding proteins. Thus the chitosan-RNA particle can comprise at least one RNA sequence complementary to or capable of binding through complementary base pairing to an mRNA sequence of at least one microsporidian mitosome protein, enzyme of microsporidian replication, microsporidian protease or microsporidian chitin-binding protein.

Table 2 provides a non-exhaustive list of some genes (and their category) of E. hepatopenaei, which can be targets (e.g. targeted gene products) for reduction of expression by the chitosan-dsRNA particle described herein.

TABLE 2
PARTIAL LIST OF CANDIDATE EHP TARGET GENE

| Seq ID | GENE | | |
|---|---|---|---|
| NO: | SEQUENCE | GENE | CATEGORY |
| 1 | EHP00_2098 | ATP-ADP carrier protein | Energy |
| | (locus tag) | | metabolism |
| 2 | EHP00_2101 | ATP-ADP carrier protein 4 | Energy |
| | (locus tag) | | metabolism |
| 3 | EHP00_1051 | Glucose-6-Phosphate | Energy |
| | (locus tag) | Dehydrogenase | metabolism |
| 4 | EHP00_1461 | Plasma membrane transporter | Energy |
| | (locus tag) | | metabolism |
| 5 | MT249228 | Polar tube protein (PTP2) 2 | Structural |
| 6 | MG015710.1 | Heparin binding protein | Structural |
| | | (EhSWP1) | |
| 7 | EHP00_1989 | ATP binding cassette (ABCG7) | Energy |
| | (locus tag) | | metabolism |
| 8 | EHP00_2562 (locus tag) | Glyceraldehyde 3 Phosphate | Energy |
| | | dehydrogenase type 1 (Gly3PD) | metabolism |
| 9 | MNPJ01000003.1:55656- | Spore Wall Protein 3 (SWP3) | Structural, |
| | 56405 | | Host-Parasite |
| | | | interaction |
| 10 | MNPJ01000003.1:55656- | Spore Wall Protein 3 (SWP3) | Structural, |
| | 56405 | Selected target sequence | Host-Parasite |
| | | | interaction |
| 11 | MN604020.1 | Spore Wall Protein 7 (SWP7) | Structural, |
| | | | Host-Parasite |
| | | | interaction |
| 12 | MN604020.1 | Spore Wall Protein 7 (SWP7)- | Structural, |
| | | Selected target sequence | Host-Parasite |
| | | | interaction |
| 13 | MNPJ01000020.1:187564- | Endochitinase | Structural |
| | 189429 | | |
| 14 | MNPJ01000020.1:187564- | Endochitinase-selected target | Structural |
| | 189429 | sequence | |
| 15 | MNPJ01000021.1:80759- | (New) Spore Wall Protein | Host-Parasite |
| | 82180 | OQS54254 | interaction |
| 16 | MNPJ01000021.1:80759- | (New) Spore Wall Protein | Host-Parasite |
| | 82180 | OQS54254-Selected target | interaction |
| | | sequence | |

It will be appreciated that each gene or sequence disclosed in Table 2 represents a separate embodiment of the invention.

In some embodiments, the RNA comprises a nucleic acid sequence which specifically reduces the gene products of a gene selected from the group consisting of a microsporidian polar tube protein 2 (PTP2) gene, a microsporidian heparin binding protein (EhSWP1) gene, a microsporidian ATP binding cassette (ABCG7) gene, a microsporidian Glyceraldehyde 3 Phosphate dehydrogenase type 1 (Gly3PD), a microsporidian spore wall protein 3 (SWP3), a microsporidian spore wall protein 7 (SWP7), a microsporidian endochitinase (Endochitinase) and a microsporidian new spore wall protein (OQS54254). In some embodiments, the microsporidian polar tube protein 2 (PTP2) gene product is encoded by SEQ ID NO: 5, or a portion thereof, the Heparin binding protein (EhSWP1) gene product is encoded by SEQ ID NO: 6, or a portion thereof, the ATP binding cassette (ABCG7) gene product is encoded by SEQ ID NO: 7, or a portion thereof, the Glyceraldehyde 3 Phosphate dehydrogenase type 1 (Gly3PD) gene product is encoded by SEQ ID NO: 8, or a portion thereof, the microsporidian spore wall protein 3 (SWP3) gene product is encoded by SEQ ID NO: 9, or a portion thereof, the microsporidian spore wall protein 7 (SWP7) gene product is encoded by SEQ ID NO: 10, or a portion thereof, the microsporidian endochitinase (Endochitinase) gene product is encoded by SEQ ID NO: 11, or a portion thereof and the microsporidian new spore wall protein (OQS54254) gene product is encoded by SEQ ID NO: 12, or a portion thereof. For example, a ss or dsRNA targeted to a microsporidian parasitic pathogen of farmed crustaceans can be a EHP-specific ss or dsRNA corresponding to any one or more of EHP sequences SEQ ID NOs: 1-16, and, in specific embodiments, any one or more of SEQ ID NOs: 5-16. Additional suitable ss or dsRNA targeted to microsporidian parasitic pathogens can be designed according to sequences from any microsporidian pathogenic in farmed aquatic animals, and, in particular, farmed crustaceans, for example, from sequences of the microsporidians infecting fish and aquatic arthropods detailed in Tables 1A and 1B.

SEQ ID NOs. 9, 11, 13 and 15 are full length coding sequences of the SWP3, SWP7, Endochitinase and “new spore wall protein”, respectively. SEQ ID NOs. 10, 12, 14 and 16 are selected target sequences of the SWP3, SWP7, Endochitinase and “new spore wall protein” sequences, respectively, specifically chosen for use in RNAi silencing of their respective gene expression. Thus, in some embodiments, the microsporidian spore wall protein 3 (SWP3) gene product is encoded by SEQ ID NO: 10, or a portion thereof, the microsporidian spore wall protein 7 (SWP7) gene product is encoded by SEQ ID NO: 12, or a portion thereof, the microsporidian endochitinase (Endochitinase) gene product is encoded by SEQ ID NO: 14, or a portion thereof and the microsporidian new spore wall protein (OQS54254) gene product is encoded by SEQ ID NO: 16, or a portion thereof.

In particular embodiments, a ss or dsRNA targeted to a microsporidian parasitic pathogen of farmed crustaceans can be a EHP-specific ss or dsRNA corresponding to any one or more of SEQ ID NOs. 5-16. In specific embodiments, the ss or dsRNA targeted to a microsporidian parasitic pathogen of farmed crustaceans can be a combination of EHP-specific ss or dsRNAs corresponding to any combination of two or more of SEQ ID NOs. 5-16. Non-limiting examples of suitable combinations of EHP-specific ss- or dsRNAs include, but are not limited to combinations comprising SEQ ID NOs. 5, 6, 7, 8, 10 and 12, combinations comprising SEQ ID NOs. 7, 8, 10, 12, 14 and 16, combinations comprising SEQ ID NOs. 5, 6, 10, 12, 14 and 16, combinations comprising any permutations of two or more of SEQ ID NOs. 5, 6, 7, 8, 9, 10, 11, 12, 13, 14, 15 and 16, and in particular, combinations comprising any permutations of two or more of SEQ ID NOs. 5, 6, 7, 8, 10 and 12.

The chitosan-ss or dsRNA particle of the invention can also be used to silence expression of endogenous host genes of the farmed aquatic animals, e.g. aquatic crustaceans. Thus, in some embodiments, the at least one RNA comprises at least one sequence binding to, at least partially complementary to or at least 90% identical to the target mRNA molecule of the farmed crustacean or farmed aquatic crustacean. Endogenous mRNAs of the farmed aquatic crustacean to be targeted using dsRNA can include, but are not limited to, those whose expression is correlated with an undesired phenotypic trait, or those whose expression is associated with susceptibility to, response to or resistance to infection by any of the microsporidian parasites pathological in farmed fish and crustaceans. Exemplary mRNAs that may be targeted are those encoding parasite recognition moieties, whose silencing would not be detrimental to the host. In other embodiments, the dsRNA is directed to a target mRNA of the host which constitutes a regulatory step of a desired process or pathway, which silencing thereof increases the activity of the pathway.

Such targets could include mRNA of negative regulators such as transcription factors and signaling pathways, mRNA of receptors responsible for downregulation of desired pathways, etc. Other desirable endogenous targets for inhibition of expression could include, but are not limited to genes and pathways responsible for enhanced growth, enhanced growth rate, improved feed conversion, targeting sex determination during development, influencing flavor, stress (temperature, salinity, toxicity, low Oxygen tension, disease, parasites, etc) resistance or tolerance, breeding behavior and even domestication of species not yet easily cultured.

Also contemplated are chitosan-RNA particles comprising multiple RNAs. Thus, in some embodiments the dsRNA of the chitosan-dsRNA particle can be homogeneous, i.e. all complementary to, binding to or at least 90% identical to the same sequence of the mRNA target molecule of the microsporidian pathogen. In addition, the ss or dsRNA of the chitosan-RNA particle can be heterogeneous—e.g. complementary to, binding to or at least 90% identical to two or more sequences of the same mRNA target molecule of the microsporidian pathogen. In other embodiments, the ss or dsRNA can comprise sequences complementary to, binding to or at least 90% identical to different mRNA target molecules of the same microsporidian pathogen. For example, in one specific embodiment the RNA of the chitosan-RNA particle described herein comprises sequences complementary to, binding to or at least 90% identical to any one, two, three or more sequences of the EHP. In some embodiments, the RNA comprises sequences complementary to, binding to or at least 90% identical to any one or more of SEQ ID Nos. 5, 6, 7, 8, 9, 10, 11, 12, 13, 14, 15 and 16. The additional sequences can thus also be complementary to, binding to or at least 90% identical to the same target mRNA, but from different segments thereof, or the additional sequences can be complementary to or binding to the different and distinct target mRNAs, from the same microsporidian pathogen.

Also contemplated are chitosan-RNA particles wherein the RNA of the chitosan-RNA particle comprises ss or dsRNA complementary to, binding to or at least 90% identical to one or more each of nucleotide sequences of mRNA target molecules of two or more distinct microsporidian pathogens. For example, in one specific embodiment, the RNA of the chitosan-dsRNA particle described herein comprises sequences complementary to, binding to or at least 90% identical to at least one each of sequences from the EHP or any other microsporidian pathogenic in farmed crustaceans. It will be appreciated that the ss or dsRNA of the particle as described herein can include sequences complementary to, binding to or at least 90% identical to all matter of combinations of target mRNAs—e.g. one or more sequences from the same target mRNA along with one or more sequences from one or more different target mRNAs. Also contemplated are chitosan-RNA particles comprising RNA sequences directed towards multiple targets. Such RNA directed towards multiple targets includes contiguous RNA sequences designed complementary to, binding to or at least 90% identical to more than one target sequence—e.g. all of SEQ ID Nos. 5-16 of the EHP comprised on the same ss or dsRNA polynucleotide, which, when processed, can produce siRNAs complementary to, binding to or at least 90% identical to multiple targets of the EHP. The advantage of such combinations as described herein is, inter alia, in the economy of production and the simplicity of formulation of the chitosan-RNA using the single ss or dsRNA.

Further, also contemplated are chitosan-RNA particles comprising ss or dsRNA complementary to, binding to or at least 90% identical to target mRNA of one or more microsporidian pathogens of more than one species of farmed crustaceans. Thus, the chitosan-dsRNA particles can comprise, for example, dsRNA complementary to, binding to or at least 90% identical to target mRNA of microsporidian parasites pathological to any one or more of Litopanaeus vannamei, Panaeus monodon, Penaeus japonicus and/or Macrobrachium rosenbergii. Still further, dsRNA may be complementary to, bind to or at least 90% identical to microsporidian pathogens of different classes of farmed crustaceans, for example, ss or dsRNA targeting microsporidian parasites of crabs as well as microsporidian parasites of lobsters. Such multiple-targeted chitosan-RNA particles can be particularly effective in an integrated, multitrophic aquaculture system, where more than one crustacean species is farmed together.

Thus, in some embodiments, the RNA comprises a nucleic acid sequence complementary to, binding to or at least 90% identical to a nucleic acid sequence of microsporidian polar tube protein 2 (PTP2) gene, a microsporidian heparin binding protein (EhSWP1) gene, a microsporidian ATP binding cassette (ABCG7) gene, a microsporidian Glyceraldehyde 3 Phosphate dehydrogenase type 1 (Gly3PD) gene, an ADP-ATP carrier protein gene, an ADP-ATP carrier protein 4 gene, a Glucose-6-phosphate dehydrogenase (ZWF1) gene, a plasma membrane transporter gene, a microsporidian spore wall protein 3 gene (SWP3), a microsporidian spore wall protein 7 (SWP7) gene, a microsporidian endochitinase (Endochitinase) gene and a microsporidian new spore wall protein (OQS54254) gene. In specific embodiments, the RNA targeting EHP comprises a ss or ds RNA complementary to, binding to or at least 90% identical to a EHP RNA sequence selected from the group consisting of a PTP2 sequence (e.g. SEQ ID NO: 5), a EhSWP1 sequence (e.g. SEQ ID NO: 6), a ABCG7 sequence (e.g. SEQ ID NO: 7), a Gly3 PD type 1 sequence (e.g. SEQ ID NO: 8), a SWP3 sequence (e.g. SEQ ID NO: 9 or SEQ ID NO: 10), a SWP7 sequence (e.g. SEQ ID NO: 1 or SEQ ID NO: 12), an endochitinase sequence (e.g. SEQ ID NO: 13 or SEQ ID NO: 14) and a new spore wall protein sequence (e.g. SEQ ID NO: 15 or SEQ ID NO: 16).

In some embodiments, the RNA targeting EHP comprises a ss or ds RNA complementary to or at least 90% identical to more than one EHP RNA sequence, for example, to a combination of two or more EHP sequences (e.g. PTP2, EhSWP1, ABCG7, Gly3 PD type 1, SWP3, SWP7, Endochitinase, new spore wall protein OQS54254). In specific embodiments, the RNA targeting EHP comprises a ss or ds RNA complementary to or at least 90% identical to a portion of a mix of EHP sequences comprising two or more of any of SEQ ID NOs. 1-16, and in particular, two or more of any of SEQ ID NOs. 5-16, (for example SEQ ID NOs. 5 and 6; 5 and 7; 5 and 8; 5 and 9; 5 and 10; 5 and 11; 5 and 12; 5 and 13; 5 and 14; 5 and 15; 5 and 16, 5 and 6 and 7; 5 and 7 and 8; 5 and 6 and 8; 5 and 6 and 10; 5 and 6 and 12; 5 and 6 and 14; 5 and 6 and 16; 6 and 7; 6 and 7 and 8; 6 and 8; 7 and 8; 9 and 10; 9 and 11; 9 and 12; 9 and 10 and 11; 9 and 10 and 11 and 12; 10 and 11; 10 and 12; 10 and 11 and 12; 11 and 12; 5 and 9 and 10 and 11 and 12; 5 and 9 and 10 and 11 and 12 and 13 and 14 and 15 and 16; and all permutations of two or more of SEQ ID NOs. 5-16, and in particular, of SEQ ID NOs. 5-8, 10, 12, 14, and 16). Some non-limiting examples of suitable permutations are permutations of any two of SEQ ID NOs. 5, 6, 7, 8, 10 and 12, permutations of any three of SEQ ID NOs. 5, 6, 7, 8, 10 and 12, permutations of any four of SEQ ID NOs. 5, 6, 7, 8, 10 and 12, permutations of any five of SEQ ID NOs. 5, 6, 7, 8, 10 and 12 and permutations of any six of SEQ ID NOs. 5, 6, 7, 8, 10 and 12.

In yet further embodiments, the chitosan-RNA particle comprises at least one additional ss or dsRNA comprising at least one additional sequence capable of binding through complementary base pairing or at least 90% identical to a target mRNA molecule of the farmed crustaceans. In other embodiments, the chitosan-RNA particle comprises at least one additional ss or dsRNA comprising at least one additional sequence at least partially complementary to or at least 90% identical to a target mRNA molecule of the farmed crustaceans.

According to yet another embodiment of the present invention, synthesis of ss or dsRNA suitable for use with the present invention can be effected according to microsporidian parasite pathogen target sequences known to be involved in the process of host cell attachment, spore germination, EHP metabolism, and/or exocytosis of the EHP spores. It will be appreciated that, in a further embodiment of the present invention, ss or dsRNA targeted to sequences having a conserved homology between different strains of the microsporidian pathogens, or even between diverse microsporidian pathogens, once such sequences are identified, can be effective against more than one variant of the microsporidian pathogen, or even against different microsporidian pathogens.

It will be appreciated from the description provided herein above, that contacting farmed aquatic animal (e.g. crustacean) cells with an RNA gene silencing agent (e.g. RNAi, ds or ss RNA) may be affected in a number of ways:
1. Transiently transfecting the host cells with the mature RNA agent;
2. Stably, or transiently transfecting the host cells with an expression vector which encodes the mature RNA agent;
3. Stably, or transiently transfecting the host cells with an expression vector which encodes a pre-RNAi. The pre-RNAi sequence may comprise from 45-90, 60-80 or 60-70 nucleotides. The sequence of the pre-RNAi may comprise a RNAi and a RNAi* as set forth herein. The sequence of the pre-RNAi may also be that of a pri-RNAi excluding from 0-160 nucleotides from the 5′ and 3′ ends of the pri-RNAi. The sequence of the pre-RNAi may comprise the sequence of SEQ ID NOS: 1-16 or variants thereof;
4. Stably, or transiently transfecting the host cells with an expression vector which encodes the pri-RNAi. The pri-RNAi sequence may comprise from 45-30,000, 50-25,000, 100-20,000, 1,000-1,500 or 80-100 nucleotides. The sequence of the pri-RNAi may comprise a pre-RNAi, RNAi and RNAi*, as set forth herein, and variants thereof.

The present invention provides methods for preparing chitosan-RNA particles as disclosed herein, comprising mixing the chitosan and intended RNA payload.

In some embodiments, the mixing of the chitosan and RNA comprises rapidly adding RNA and chitosan solutions while rapidly agitating (e.g. approximately 1500 rpm), and rapidly agitating the mixture for 1-10, 1-5, 1, 2, 3, 4, 5, 6, 7, 8, 9 or 10 minutes (e.g. approximately 1500 rpm). In particular embodiments, the chitosan solution comprises 2-20 mg/ml low molecular weight chitosan in mild acidic buffer. In particular embodiments, the chitosan solution is 3 mg/ml. In specific embodiments, the buffer is acidified with an organic acid. In some embodiments, the organic acid is selected from acetic acid, lactic acid, propionic acid and citric acid. In specific embodiments, the chitosan solution is 3 mg/ml low molecular weight chitosan in 0.02 M citric acid buffer, pH 4-5. In other embodiments, the chitosan solution is 3 mg/ml low molecular weight chitosan in 0.02 M citric acid buffer, pH 4-5.

In some embodiments, the pH of the chitosan-RNA complex is pH 3.5-5.0. In other embodiments, the chitosan-RNA particle has a pH of 3.5, 3.6, 3.7, 3.8, 3.9, 4.0, 4.1, 4.2, 4.3, 4.4, 4.5, 4.6, 4.7, 4.8, 4.9 or 5.0.

In some embodiments, the RNA solution comprises 1-5 mg/ml RNA in water. In specific embodiments, the RNA solution is 3 mg/ml RNA in water. In still further embodiments, the RNA is provided in a solution of a sodium salt such as sodium chloride, sodium sulfate or sodium citrate. Inasmuch as the presence of chloride ions is less desirable for scaled-up industrial processes, in some embodiments the sodium salt is a non-chloride sodium salt, e.g. sodium citrate or sodium sulfate.

In some embodiments, following agitation, the mixture is desalted and concentrated. In some embodiments, following desalting and concentration, the chitosan-RNA complex solution is further dried to produce the chitosan-RNA particle. In some embodiments the chitosan-RNA complex solution is dried using for example, but not exclusively, a spray dryer, a belt dryer, a drum filter, a Nutche filter and the like. Following the drying step, in some embodiments the dried chitosan-RNA particle composition is ground to approximate the size of shrimp feed particles (micron grade).

In specific embodiments, the method for producing the chitosan-RNA particle composition comprises:
i) rapidly adding an RNA solution to 50-100% deacetylated low molecular weight chitosan solution, wherein said chitosan solution comprises 3 mg/ml low molecular weight chitosan in 00.1-0.01 M organic acid, in a ratio of 1:1 volumes of chitosan to RNA.
(ii) desalting and concentrating the chitosan-RNA complex solution, and
(iii) drying the desalted, concentrated chitosan-RNA complex solution.

Following drying, the chitosan-RNA particle composition can be used immediately, or stored until use. In some embodiments, the dried chitosan-RNA particle composition can be stored until mixing with other components to form a pre-mix which can be provided to a feed production facility, for preparation of the feed comprising the chitosan-RNA particle composition or nutriceutical.

Chemical modifications of chitosan have been pursued to enhance its gene transfection efficiency. Most commonly, chitosan can be structurally modified with hydrophobic, hydrophilic, amphiphilic, CPPs, and cell specific ligands using the reactive amino group (C2 position) and hydroxyl groups (C6 and C3 positions).

The chitosan-RNA particles of the invention can also be prepared with chemical modifications (in addition to reduction of degree of deacetylation described above), for example, to enhance the effectiveness of binding and delivery of the ss or dsRNA at the target cell. According to some embodiments, the chitosan-RNA particle further comprises a polymer. A non-limiting list of polymers suitable for addition to the chitosan-RNA particle of the invention includes poly(lysine) (PLL), linear polyethyleneimine (1-PEI), branched polyethyleneimine (b-PEI), poly(ethylene glycol) (PEG), G3 dendritic poly(amido amine) (PAMAM), linear poly(amino amine) (PAA), poly(lactide-co-glycolide) (PLGA) and poly (beta-amino ester) (PBAE).

In some embodiments, the chitosan-RNA particle is a stable particle. As used herein, the term “stable chitosan-RNA particle” is defined as a particle which maintains a significant portion of the RNA “payload”, even under difficult conditions. One suitable measure of stability is the ability to maintain a majority of the initial RNA “payload”. Thus, in some embodiments, the chitosan-RNA particle is a stable particle, retaining at least 60% of the RNA of the complex when exposed to an aqueous environment (e.g. seawater, freshwater, brackish water, etc). In other embodiments, the chitosan-RNA particle retains at least 60%, at least 70%, at least 80%, at least 90% or more of the RNA of the complex when exposed to an aqueous environment or medium. In specific embodiments, the chitosan-RNA particle retains at least 60%, at least 70%, at least 80%, at least 90% or more of the RNA of the complex when exposed to an aqueous environment at a pH range of 4.2-8.0. In some embodiments, the pH of the aqueous environment or medium is in the range of 6.5-7.8, or, more specifically, pH 7.5.

The particulate compositions and/or chitosan-dsRNA particles as disclosed can be used for treating or preventing disease in farmed aquatic animals, such as fish and aquatic crustaceans. The disclosed particles, or compositions comprising the particles can be provided or administered to the farmed animals in a variety of methods of administration, including feeding, contact with gill tissue, via the eyes, via the skin or cuticle (particularly important during a molt) and via external wounds (cuts or abrasions). Parenteral administration of compositions comprising the particles as disclosed is also contemplated.

In particular embodiments, the disclosed particulate compositions and/or chitosan-RNA particle can be provided (e.g. administered) as a nutriceutical composition, comprising farmed aquatic animal food and the particulate compositions or chitosan-RNA particles disclosed herein. In some embodiments, the nutriceutical composition comprises particles comprising ss or dsRNA of a single sequence, i.e. all directed to a single target RNA sequence. In other embodiments, the nutriceutical comprises chitosan-RNA particles comprising ss or dsRNA directed to more than one target mRNA sequence, as detailed herein. In all, it will be noted that the nutriceutical can comprise any particulate compositions, chitosan-RNA particle, or any combination thereof described or suggested herein.

As used herein, the term “nutriceutical composition” is a combination of “nutritional composition” and “pharmaceutical composition” and refers to an ingestible substance that has one or more beneficial effects on an organism such as a farmed fish and/or crustacean. The term “nutriceutical” can also refer to one or more compounds which are present in an ingestible substance. Ingestible substances include, but not limited to dietary supplements, foods and the like. The terms “nutriceutical” and “nutritional supplement” may be used interchangeably. A substance (e.g., a food product, a nutriceutical product or a pharmaceutical) having beneficial biological, nutriceutical or medicinal properties refers to the ability of the substance to provide an individual one or more health benefits as described herein (e.g., in the resistance to, prevention, reduction and/or cure of one or more diseases and/or disorders described herein).

The nutriceutical composition can be administered to treat a disease or silence a gene, and examples of farmed crustaceans to be treated and diseases that can be prevented or cured in the treated crustaceans are described herein. Depending on the farmed crustacean to be treated the formulations can be administered either by providing the chitosan-RNA particles with the feed of the fish or crustacean or, in the case of some shrimp or other crustaceans, by providing it in the water in which the animal lives. It will be appreciated that chitosan is derived from chitin, which constitutes a significant portion of the diet of many marine and fresh water species. Yet further, marine crustaceans are known to be cannibalistic, feeding off members of their own communities as well as non-related populations. Thus, the chitosan-RNA particles described herein may also be effective in delivering the ss or dsRNA to the farmed crustacean without combination with feed, provided in the water of the aquaculture environment in which the organism lives.

It will be appreciated that the therapeutic effects of the nutriceutical, when provided as feed, or along with feed in the water of a farmed crustacean subject, may be dependent upon ingestion by the crustacean subject, but may also be mediated by contact with the tissues of the subject crustacean, for example, contact with the gills, eyes or other surfaces which may allow transfer of the particulate compositions or particles described herein, e.g. whole particles, or even just the ss or dsRNA component of the particles to cells of the host crustacean subject.

The particles may be introduced into the aquatic animal (e.g. fish or crustacean), with a physiologically acceptable vehicle and/or adjuvant. Useful vehicles are well known in the art, and include, e.g., water, buffered water, saline, glycine, hyaluronic acid and the like. The resulting aqueous solutions may be packaged for use as is, or lyophilized, the lyophilized preparation being rehydrated prior to administration, as mentioned above. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, and the like. In one embodiment of the invention, the particle is encapsulated such that the resulting composition is water resistant but amenable to release within the digestive system of the crustacean host. In another embodiment, the molecule is combined with a binder that assists in associating the molecule with feed, which is particularly useful for oral administration. Such a water resistant binding substance can be any substance having such properties. Examples include, without limitation, agarose or other sugar compounds, albumin, alginate or any similar composition.

The compositions of the invention can be provided in feed. Feeds for farmed aquatic species include fish oils and proteins as well as plant proteins, minerals, and vitamins that achieve the nutrition requirements of the fish or crustaceans and may also offer health benefits to humans (consuming the farmed species). Traditionally, diets for fish contained 30-50% fish meal and oil. Feed suitable for feeding farmed aquatic fish, crustaceans and mollusks can be similar or different in formulation. For example, fish can be fed various foodstuffs including but not limited to fishmeal, pea seed meal, wheat bran, wheat flour, blood meal, vitamins, corn gluten and wheat gluten.

Crustaceans can be fed various foodstuffs including but not limited to soy bean oil cake, fish meal, fish oil, wheat flour, soybean meal, squid oil, Brewer's yeast, shrimp meal, squid meal, alfalfa, wheat gluten, squid liver powder, yeast, shrimp head meal, shrimp shell meal and vitamins. These can be provided in many forms, for example, as feed pellets.

The composition of some embodiments of the invention may further comprise at least one of a surface-active agent, an inert carrier vehicle, a preservative, a feeding stimulant, an attractant, an encapsulating agent, a binder, an emulsifier, a dye, an ultra-violet protector, a buffer, a flow agent or micronutrient donors (e.g. vitamins), a drug or other preparations that influence the health or growth, well being or stress/disease tolerance of the farmed crustacean.

The particulate compositions and/or chitosan-RNA particles of the invention can be prepared as a nutriceutical for farmed crustaceans by mixing the particles with feed of the farmed crustaceans. The particulate compositions and/or chitosan-RNA particles of the invention can be mixed with the farmed crustacean food in different ways, for example, the particles can be added to the shrimp feed by mixing the formulation with readymade feed and air drying, then spraying the mixture with fish oil or gelatin. In some embodiments, the particulate compositions and/or chitosan-RNA particles can be mixed with the feed as a formulation (liquid) and then the feed top sprayed with oil or gelatin. The feed can also be top-sprayed with both gelatin and the liquid formulation of chitosan-RNA particles. Dry powder formulations of the particulate compositions or chitosan-RNA particles can also be used, for example, by spray drying the chitosan-RNA particles, mixing the spray dried chitosan-RNA with the feed ingredients and top spraying with oil or gelatin.

In some embodiments, the particles are mixed with the feed at a ratio of about 0.5-10% particles (w/w) in the feed. In some embodiments, the particles are mixed with feed at 0.75-7.5%, 1.0-5.0%, 1.5-4.5%, 2-8%, 1%, 2%, 3%, 4%, 5% or 6%. The particles can also be provided according to dosage of “payload” RNA per gram crustacean feed, for example, 210-10,000 μg ss or dsRNA per gram of shrimp and/or prawn feed, 50-8,000 μg ss or dsRNA per gram of shrimp and/or prawn feed, 100-5,000 μg ss or dsRNA per gram of shrimp and/or prawn feed, 200-1000 μg ss or dsRNA per gram of shrimp and/or prawn feed, 250-5,000 μg ss or dsRNA per gram of shrimp and/or prawn feed, and 500-5,000 μg ss or dsRNA per gram of shrimp and/or prawn feed. It will be appreciated that each individual range of particles or dsRNA per gram feed ratio represents a single, separate embodiment. In a specific embodiment, the particles are mixed with the feed at a ratio of 0.1 to 50 mg ss or dsRNA per gram of shrimp and/or prawn feed. In particular embodiments, the particles or particulate composition are mixed with the feed at a ratio of about 0.1 mg/g feed, 0.5 mg/g feed, 1.0 mg/g feed, 2.0 mg/g feed, 4.0 mg/g feed, 5.0 mg/g feed, 7.5 mg/g feed, 8 mg/g feed, 10 mg/g feed, 12 mg/g feed, 15 mg/g feed, 17.5 mg/g feed, 20 mg/g feed, 22 mg/g feed, 25 mg/g feed, 27.5 mg/g feed, 28 mg/g feed, 30 mg/g feed, 32 mg/g feed, 34 mg/g feed, 36 mg/g feed, 40 mg/g feed, 42 mg/g feed, 44 mg/g feed, 46 mg/g feed, 48 mg/g feed or 50 mg/g feed.

In some embodiments, the nutriceutical composition is defined according to the RNA content (w/w) of the composition. It will be appreciated that the RNA content of the nutriceutical relates to the % of the “payload” ds or ss RNA contributed by the chitosan:RNA particles, and does not include RNA contributed by the feed or any other component of the nutriceutical.

In particular embodiments, the RNA content of the nutriceutical composition is in the range of 0.01-10% RNA (w/w). In other embodiments, the RNA content is in the range of 0.05-5.0%, 0.1-3.0%, 0.25-2.5%, 0.5-2%, 0.1-0.5% (w/w) or intermediate ranges. In specific embodiments, the RNA (w/w) content of the nutriceutical is 0.05, 0.1, 0.15, 0.2, 0.25, 0.3, 0.35, 0.4, 0.45, 0.5, 0.55, 0.6, 0.65, 0.7, 0.75, 0.8, 0.85, 0.9, 0.95, 1.0, 1.25, 1.5, 1.75, 2.0 or 2.5%. In yet further embodiments the RNA content (w/w) of the nutriceutical composition is in the range of 0.1-0.5%.

In further embodiments, provision of the particle or nutriceutical composition may be varied throughout the duration of culturing of the crustacean, or during portions thereof. For example, in some embodiments, the particles or particulate composition is provided in the feed for 7 days at 50 mg ss or ds RNA per gram feed, or for 21 days at 0.1 to 1 mg ss or dsRNA per gram feed. It will be understood that regimens can vary over the life of the farmed crustacean, and that various RNA/feed ratios can be implemented at different times and for a variety of durations during the culturing of the farmed crustaceans. Likewise, in some embodiments, administration of the articulate composition or nuctaceutical can be initiated upon detection of infection or risk of infection, and in other embodiments, administration can be prophylactic, anticipating and attempting to prevent or attenuate infection.

In some embodiments, wherein the crustacean is shrimp and/or prawns, the particulate composition and/or nutriceutical composition is provided in a dosage of 3-10% of the shrimp's body weight per daily feeding. In specific embodiments the particle or nutriceutical composition of the invention is provided in a dosage of 5% of the shrimp's body weight per feeding. It will be appreciated that the amount of nutriceutical composition provided to the shrimp can vary with the shrimp's body weight. For example, in some embodiments, some younger shrimps (e.g. <0.50 g body weight), are fed up to 400% of the shrimp's body weight. In more mature shrimp (e.g. >7 g body weight), for example, feed is provided up to 7% of their body weight.

Thus, in particular embodiments, young shrimp, having body mass (weight) between 3-30 milligrams are provided with feed (e.g. the nutriceutical) equaling 90-400% of the body mass per day. In some embodiments, young shrimp, having body mass (weight) between 3-30 milligrams are provided with feed (e.g. the nutriceutical) equaling 90, 100, 120, 140, 160, 180, 200, 220, 240, 260, 280, 300, 320, 340, 360, 380 or 400% of the body mass per day.

In particular embodiments, shrimp having body mass (weight) between 30-milligrams to 3 grams are provided with feed (e.g. the nutriceutical) equaling 7-90% of the body mass per day. In some embodiments shrimp, having body mass (weight) between 30 milligrams and 3 grams are provided with feed (e.g. the nutriceutical) equaling 7, 10, 15, 20, 25, 30, 35, 40, 45, 50, 55, 60, 65, 70, 75, 85 or 90% of the body mass per day.

In still other embodiments, grown shrimp having body mass (weight) between 3-7 grams or greater are provided with feed (e.g. the nutriceutical) equaling 3-7% of the body mass per day. In some embodiments shrimp, having body mass (weight) between 3-7 grams (or greater) are provided with feed (e.g. the nutriceutical) equaling 3, 3.5, 4, 4.5, 5, 5.5, 6, 6.5 or 7% of the body mass per day.

Frequency of provision of the particle or nutriceutical composition (regimen) will vary with the farmed aquatic species, and, in some cases, with the specific stages of the life cycle of the farmed organisms. Thus, in some embodiments, the farmed organisms is shrimp and/or prawn, and the particle or nutriceutical of the invention is provided in 1-10, 2-8, 3-6 or 1-3 feedings per life cycle of the shrimp and/or prawns. In a particular embodiment, the farmed crustacean is shrimp and/or prawns and the particle or nutriceutical of the invention is provided in 1-3 feeding per life cycle of the shrimp and/or prawns. In another embodiment, each of the feedings is at least once, twice, at least three times, at least four, five or more times per day, and each feeding period extends for at least two, at least three, at least four, five or more days. In some particular embodiments, each of the feedings is at least three times per day. In another embodiment, each feeding period extends for at least five days.

In particular embodiments, feeding is provided on a daily basis, and the “dosage” of the nutriceutical or chitosan-RNA particle is provided in feedings of 2-5 times per day. In further embodiments, the farmed crustaceans are shrimp or prawns, and the feeding is performed 5 times per day.

The invention therefore provides methods of treating or preventing a microsporidian parasitic disease or silencing a gene by administering to a farmed crustacean the chitosan-RNA particle, formulation or nutriceutical composition of the invention. The chitosan-RNA particle, formulation or nutriceutical composition can be administered by any manner described herein. The particulate compositions, particles or nutriceutical of the invention are also useful in the manufacture of a medicament for the treatment of microsporidian diseases or for silencing microsporidian genes, as described herein. A non-limiting listing of microsporidian parasites of farmed fish and crustaceans, suitable for treatment with the compositions and methods of the invention, is provided herein (see Tables 1A and 1B).

In some embodiments, ingestion of the particulate compositions, particle or nutriceutical composition of the invention by the farmed fish or crustacean results in reduction in the level of the at least one gene product of the pathogenic microsporidian in the farmed fish or crustaceans, compared to the level of the pathogenic microsporidian gene product in at least one of the same farmed fish or crustaceans ingesting feed devoid of the ss or dsRNA targeted to the gene product of the pathogenic microsporidian. In other embodiments, ingestion of the chitosan-RNA particle, formulation or nutriceutical composition of the invention by the farmed fish or crustacean results in reduction in the level of the pathogenic microsporidian in the farmed fish or crustaceans, compared to the level of the pathogenic organisms in at least one of the same farmed fish or crustaceans ingesting feed devoid of the ss or dsRNA targeted to the gene product of the pathogenic microsporidian.

With the chitosan-RNA particle, formulation or nutriceutical compositions of the present invention, it is possible to achieve protection against microsporidian disease in a farmed fish or crustacean for long periods. Protection periods after at least one feeding with the particles or nutriceutical composition targeting the pathogenic microsporidian of at least two weeks, at least 20 days, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60 days or more, can been achieved using the invention. It will be appreciated that each individual period represents a single, separate embodiment.

In some embodiments, ingestion by, or contact with the farmed fish or crustacean, of the particulate composition, formulation or nutriceutical composition results in increased survival, yield, growth rate, vigor, biomass, feed conversion, size, quality of taste and odor or stress tolerance of said farmed fish or crustaceans, compared to said farmed fish or crustaceans ingesting feed devoid of ss or dsRNA targeted to a gene product of said pathogenic microsporidian.

In US 20190175518 to Ufaz et al, the inventors have shown that administration of chitosan-ssRNA particles as well as chitosan-dsRNA particles comprising RNA sequences targeted to shrimp WSSV viral sequences, are effective in protecting shrimp against infection and the pathologies of lethal WSSV infection. The chitosan-RNA particles or nutriceutical composition of the present invention have also been shown to be highly effective delivery vehicles for administering gene silencing RNA sequences to aquatic crustaceans. Thus, in some embodiments, ingestion by, or contact with the farmed crustacean, of the chitosan-RNA particles or nutriceutical of the present invention results in increased survival of said farmed crustaceans, compared to said farmed crustaceans ingesting feed devoid of, or not coming in contact with ss or dsRNA targeted to a gene product of said pathogenic microsporidian. As used herein, the term “survival” is defined as maintenance of viability. Lack of “survival” is indicated by death.

As used herein, the terms “microsporidian pathology” or “pathogenic microsporidian infection” is defined as undesirable changes in the physiology, morphology, reproductive fitness, economic value, vigor, biomass, taste quality, odor, stress-tolerance of a farmed fish or crustacean, directly or indirectly resulting from contact with a farmed fish or crustacean pathogenic microsporidian. According to one embodiment of the invention, the undesirable changes include, but are not limited to biomass and/or yield of the diseased or pathogen infected fish or crustacean. According to another embodiment of the invention, change in yield includes, but is not limited to change in yield per volume of aquaculture, change in quality of the farmed fish or crustaceans, taste quality and odor quality (e.g. of the flesh) and the like. In some embodiments, the host crustacean is a shrimp, prawn or crayfish and the pathogenic microsporidian is Enterocytozoon hepatopenaei (EHP). In some embodiments, the host crustacean is infected with Hepatopancreatic microsporidiosis.

Clinical signs of Hepatopancreatic microsporidiosis (EHP infection) include growth retardation, in particular, and an unusually sudden retarded growth, in the absence of other gross signs of disease. Microscopic pathological signs of EHP infection can include hepatopancreas tubule epithelial cells showing basophilic, cytoplasmic inclusions (microsporidian sporocysts) containing clusters of elliptical to ovoid spores of 1.1 by 0.6-0.7 μm in tissue sections stained with H&E; and sometimes the observation of free spores released from infected cells, in the lumen of the tubuli.

Other signs accompanying the microsporidian infection may be reduction in food consumption and lethargy.

As used herein, the phrase “stress tolerance” refers to both tolerance to biotic stress, and tolerance to abiotic stress. The phrase “abiotic stress” as used herein refers to any adverse effect on metabolism, growth, viability and/or reproduction of a host fish or crustacean caused by a-biotic agents. Abiotic stress can be induced by any of suboptimal environmental growth conditions such as, for example, poor oxygenation, high concentrations of toxins, pollutants or waste in the water, low or high water temperature, heavy metal toxicity, high or low nutrient levels (e.g. nutrient deficiency), high or low salt levels (e.g. salinity), atmospheric pollution, high or low light intensities (e.g. insufficient light) or UV irradiation. Abiotic stress may be a short term effect (e.g. acute effect, e.g. lasting for about a week) or alternatively may be persistent (e.g. chronic effect, e.g. lasting for example 10 days or more). The present disclosure contemplates situations in which there is a single abiotic stress condition or alternatively situations in which two or more abiotic stresses occur.

As used herein the phrase “abiotic stress tolerance” refers to the ability of a farmed fish or aquatic crustacean to endure an abiotic stress without exhibiting substantial physiological or physical damage (e.g. alteration in metabolism, growth, viability and/or reproducibility of the crustacean).

According to some embodiments, ingestion or contact with the particle or nutriceutical of the invention increases growth rate and feed conversion. Growth rate can be measured by biomass or yield, and can be used to feed conversion. As used herein, the phrase “feed conversion” refers to a measure of farmed fish or crustacean production per unit of nutrient provided. Feed conversion efficiency is typically a result of an alteration in at least one of the uptake, spread, absorbance, accumulation, relocation (within the crustacean) and use of nutrients absorbed by the fish or crustacean.

As used herein the term/phrase “biomass”, biomass of an aquatic farmed species or “host crustacean biomass” refers to the amount (e.g., measured in grams of air-dried or wet tissue) of a tissue produced from the host organism in a growing season.

As used herein the term/phrase “vigor” refers to the amount (e.g., measured by weight) of tissue produced by the farmed aquatic fish or crustacean in a given time. Increased vigor could determine or affect the yield or the yield per growing time or growing area.

As used herein the term/phrase “yield” refers to the amount (e.g., as determined by weight or size) or quantity (e.g., numbers) of tissues or organs produced per individual farmed fish or crustacean, a population of the farmed fish or aquatic crustacean or per growing season. Increased yield can affect the economic benefit one can obtain from the aquaculture in a certain growing area and/or growing time.

According to one embodiment the yield is measured by protein content.

As used herein “biotic stress” refers stress that occurs as a result of damage done to the farmed crustaceans by other living organisms, such as bacteria, viruses, fungi, parasites, beneficial and harmful insects.

In some embodiments of the invention, ingestion of or contact with the nutriceutical or particle of the invention results in: improved tolerance of abiotic stress (e.g., tolerance of poor water quality, heat, cold, non-optimal nutrient or salt levels) or of biotic stress (e.g., crowding or wounding); a modified primary metabolite (e.g., fatty acid, oil, omega-3 oils, amino acid, protein, sugar, or carbohydrate) composition; a modified secondary metabolite (e.g., alkaloids, terpenoids, polyketides, non-ribosomal peptides, and secondary metabolites of mixed biosynthetic origin) composition; a modified trace element (e.g., iron, zinc), or vitamin (e.g., tocopherols) composition; improved yield (e.g., improved yield under non-stress conditions or improved yield under biotic or abiotic stress); improved ability to use nutrients; modified growth or reproductive characteristics; improved harvest, storage, or processing quality (e.g., improved harvest, storage, or processing quality), improved appeal to consumers; or any combination of these traits.

As used herein the term “improving” or “increasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater increase in any of the abovementioned parameters, as compared to the same or similar farmed fish or crustaceans infected with the same pathogen or having the same disease, and not ingesting or contacting the nutriceutical or particle or composition (i.e., farmed aquatic crustacean not contacted with or ingesting the nutriceutical or chitosan-RNA particle) of the disclosure.

As used herein the term “decreasing” refers to at least about 2%, at least about 3%, at least about 4%, at least about 5%, at least about 10%, at least about 15%, at least about 20%, at least about 25%, at least about 30%, at least about 35%, at least about 40%, at least about 45%, at least about 50%, at least about 60%, at least about 70%, at least about 80%, at least about 90% or greater decrease in disease or pathology signs such as growth retardation and the like of the farmed fish or crustacean, as compared to the same or similar untreated fish or crustacean.

In some embodiments, the changes in the negative or positive parameters of the treated farmed fish or crustaceans is measured at a time point 2-3 weeks post treatment, 3-4 weeks post treatment, 5-7 weeks post treatment, 1-2 months post treatment, 2-4 months post treatment, 4-6 months post treatment, 5-8 months post treatment and 5-12 months post treatment or more.

According to some embodiments of the invention, health/disease parameters are monitored in the treated fish or crustaceans following ingestion or contact with the nutriceutical or particle. In some embodiments, monitoring of the parameters (of gene expression and/or tolerance to stress, growth rate, etc) can be used to determine regimen of treatment of the fish or crustaceans, for example, additional introduction of the nutriceutical or particle of the invention, augmentation of the treatment with other treatment modalities (e.g. pestcticide, antibiotics, additional species for multi-species aquaculture, etc). Selection of individuals for monitoring in a tank or pen can be random or systematic (for example, sentinel fish or crustaceans can be pre-selected prior to the treatment).

Also provided by the present invention are farmed fish or crustaceans comprising the chitosan-RNA particle or the nutriceutical composition described herein. Any farmed fish or crustaceans (e.g. shrimp or prawns) can comprise the particle or nutriceutical composition, and in particular, any one of the group consisting of Shrimp, Prawns, Crabs, Lobsters and Crayfishes. In some specific embodiments the farmed crustacean is a shrimp or prawn. Suitable shrimps or prawns can be selected from the group consisting of Litopanaeus vannamei, Panaeus monodon, Penaeus japonicas and Macrobrachium rosenbergii.

Yet further, in some embodiments there is provided an aquaculture environment comprising the farmed fish or crustacean comprising the particles or nutriceutical compositions described herein.

The term “farmed crustacean” or “farmed fish” as used herein refers to a fish or crustacean which is either strictly or partially aquatic (i.e. living at least a portion of the organism's life cycle in water), and which is cultivated (i.e. grown) by man, in an aquaculture environment. Aquaculture environments can include man-made tanks, man-made ponds, cages, pens and other types of enclosures. The term “aquaculture environment” as used here is synonymous with the term “aquaculture system”. The following describes aspects and different types of aquaculture environments suitable for use with the compositions and methods disclosed herein.

One type of aquaculture environment is mariculture, a branch of aquaculture that cultivates marine organisms either in the open ocean, an enclosed portion of the ocean, or tanks or ponds filled with seawater. Finfish (e.g. flounder and whiting), marine crustaceans (e.g. prawns, shrimp, lobsters and crabs) and marine mollusks (e.g. oysters and abalone) can be cultured in seawater. Fresh water aquaculture is suitable for fresh water species, including fish (e.g. tilapia, trout), crustaceans (e.g. crayfish) and fresh water mollusks (e.g. clams). Some species are particularly suited for culture in brackish water (carp, catfish).

Some aquaculture systems employ running water, to provide oxygenated water and eliminate waste products. Other aquaculture environments, without access to affordable running water recycle part, or all of the water of the enclosures, while still others employ various techniques (spraying, paddles) to aerate the standing water of the enclosure. Some aquaculture systems use water treatment systems such as remediation (e.g. bio-remediation) systems, for example, the re-circulating system described in U.S. Pat. No. 8,506,881 to Bradley, et al.

Aquaculture environments can include open pond systems, such as ponds, pools, pens and lakes, where the water surface is exposed to the open air, or closed-pond systems, in which the pond is covered, allowing better control of sunlight, temperature and gases.

Some aquaculture systems are monoculture systems—a single species of fish or crustacean is farmed within the entire aquaculture environment. Monoculture has advantages (ease of harvest, simple nutritional requirements), but also disadvantages, as farming of a single species does not allow recycling of nutrients. Integrated multitrophic aquaculture (IMTA), on the other hand, cultivates a number of different species together within the same aquaculture environment, and so the nutritional needs of the component species can be combined into the single system which recycles nutrients (e.g. fish and crustacean waste provides nutrients for mollusks and seaweed, when cultivated together).

In some embodiments, the farmed crustaceans suitable for use with the methods and compositions of the present invention are aquatic shrimps, prawns and the like. In some embodiments, farmed crustaceans suitable for use with the invention disclosed herein are selected from the group consisting of Shrimp, Prawns, Crabs, Lobsters and Crayfishes. In particular, in some embodiments the farmed crustaceans are shrimps and/or prawns. Specifically, in some embodiments, the shrimp or prawns are selected from the group consisting of Litopanaeus vannamei, Panaeus monodon, Penaeus japonicus and Macrobrachium rosenbergii.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

It is understood that any Sequence Identification Number (SEQ ID NO) disclosed in the instant application can refer to either a DNA sequence or a RNA sequence, depending on the context where that SEQ ID NO is mentioned, even if that SEQ ID NO is expressed only in a DNA sequence format or a RNA sequence format. For example, SEQ ID NO: 5 (PTP2) is expressed in a DNA sequence format (e.g., reciting T for thymine), but it can refer to either a DNA sequence that corresponds to a PTP nucleic acid sequence, or the RNA sequence of an RNA molecule nucleic acid sequence. Similarly, though some sequences are expressed in a RNA sequence format (e.g., reciting U for uracil), depending on the actual type of molecule being described, it can refer to either the sequence of a RNA molecule comprising a dsRNA, or the sequence of a DNA molecule that corresponds to the RNA sequence shown. In any event, both DNA and RNA molecules having the sequences disclosed with any substitutes are envisioned.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

### Example 1: Effective Prevention of EHP Infection with RNAi

### Bioinformatic Analysis and Selection of Gene Targets

To evaluate the effect of dsRNA on microsporidian propagation, it was decided to target essential EHP genes that are responsible for cell invasion and energy parasitism. dsRNA sequences for the targeted genes were designed by optimization RNAi target-sites by ranking sequences according to their predicted specificity, efficiency and complexity using the online service https://www(dot)dkfz(dot)de/signaling/e-rnai3/and were synthesized commercially by from Genolution http://genolution(dot)co(dot)kr/agrorna/service-overview/.

### Polar Tube Protein 2

A polar tube is an invasion apparatus of microsporidia and is composed of six polar tube proteins (PTPs). PTP-2 (accession number MT249228.1) is a conserved protein among microsporidia and localized on the whole polar tube of germinated spores.

### Heparin Binding Protein (EhSWP1)

EhSWP1 (MG015710.1) is an important host-parasite interaction protein, located in the walls of purified, mature spores. The protein is involved in tethering spores to host-cell-surface heparin during the process of infection.

### ATP Binding Cassette ABCG7

The NoboABCG1.1 cassette was first identified in N. Bombycis, an obligate intracellular parasite infecting Bombyx mori (silkworms). The NBD domain of NoboABCG1 towards the host cytoplasm suggesting on its role in ATP binding and hydrolyzing host ATP. The protein is expressed during all the infection life stages of the parasite suggesting its importance during substrate transport. Knockdown expression of NoboABCG1 by dsRNA, significantly inhibited propagation of N. Bombycis. ABCG7 protein in EHP microsporidian (EHP00_1989) was identified as NoboABCG1 homolog.

### Glyceraldehyde-3-Phosphate Dehydrogenase Type I (Gly3PD)

EHP microsporidian cannot generate ATP by either glycolysis or oxidative phosphorylation since the parasite possesses only two of the ten enzymes in the glycolytic pathway. One of the enzymes is glyceraldehyde 3-phosphate dehydrogenase which is a key enzyme in the glycolytic pathway that catalyzes the conversion of D-glyceraldehyde 3-phosphate to 1,3-diphosphoglycerate. Knockdown of this gene might significantly affect parasite propagation.

### Experimental Methods

### Treatment Preparations

Synthetic dsRNAs were purchased from Genolution Inc, Republic of Korea.

### Injection Preparation

dsRNA molecules were purified using isopropanol precipitation and quantified by RP-HPLC. The injection cocktail was prepared by mixing the four molecules to the final concentration of 1 μg of dsRNA per 1 gram shrimp (of each molecule), in total equaling 4 μg dsRNA per 1 gram of shrimp (1 μg of each of PTP2, EhSWP1, ABCG7 and Gly3PD dsRNA).

### Feed Preparation

dsRNA comprising molecules targeting all four of PTP2, EhSWP1, ABCG7 and Gly3PD were mixed with chitosan at a ratio of 2:1 (dsRNA, chitosan). The complexes were characterized in terms of zeta potential, size, complex integrity, and dsRNA concentration in the complex (Table 3). The complex was then dried, and the resultant dry powder mixed with the shrimp feed to reach the desired dsRNA concentration in the feed. The concentration of each dsRNA was ~2%, and the total concentration of dsRNA in the feed was 7.63% (w/w). dsRNA concentration in the feed was validated by RP-HPLC analysis.

### Challenge Test

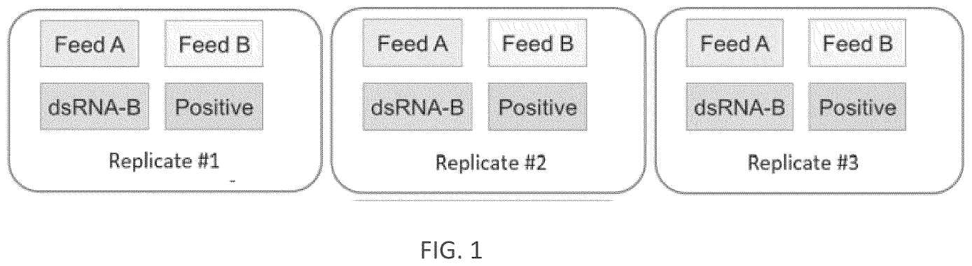

The EHP challenge test was performed in triplicates, each experimental replicate (FIG. 1) was performed in a 500-L tank containing 200-L artificial seawater. In each of the square tanks, shrimp (~2 g weight) were acclimatized in four groups of 15 animals for two days before the test. Each group of shrimps represented a different treatment:
Treatment 1—Shrimps were fed feed A containing chitosan-encapsulated dsRNA targeting the PTP2 gene;
Treatment 2—Shrimps were fed feed B containing a mix of chitosan-encapsulated dsRNAs targeting each of the four EHP target genes (PTP2, EhSWP1, ABCG7 and Gly3PD);
Treatment 3—Shrimps were injected with a cocktail of dsRNAs targeting each of the four EHP target genes (PTP2, EhSWP1, ABCG7 and Gly3PD), at a concentration of 1 μg of each dsRNA per gram shrimp, and
Treatment 4—positive control—shrimp fed feed without the addition of dsRNA and without injected dsRNA.

Pre-feeding: Pre-feeding was initiated six days before infection with EHP. Treatment groups 1 and 2 were fed feeds A (dsRNA targeting PTP2 only) and B (dsRNA targeting all of PTP2, EhSWP1, ABCG7 and Gly3PD), respectively, while the injection group (Treatment 3) received positive control feed (no dsRNA in feed).

Shrimps of Treatment 3 were injected intramuscularly with the dsRNA in 50 μl volume one day after the infection.

### EHP Infection

EHP infection was initiated by the addition of five verified infected shrimps (10-15 g) to the tank containing the prefed shrimp. On days 7 and 12, 5 shrimps from each replicate were randomly selected for hepatopancreas (HP) collection. DNA from the HP tissue samples was extracted, and EHP copy number in the samples was quantified by qPCR. A standard curve was constructed in order to quantify the EHP copy number in the samples, using a recombinant plasmid with a spore wall protein 1 (SWP1) insert. The EHP copy numbers were calculated based on the standard curve and significant differences in the copy numbers among each treatment were evaluated by the One-Way ANOVA test.

FIG. 1 illustrates the experimental design of the challenge.

The large squares each represent a 500-L tank, filled with 200-L of artificial seawater. Each tank contains replicates of the four treatments (Treatments1-4). The smaller rectangles represent baskets containing 15 shrimp each.

### Results

### Treatment Preparations

Chitosan-dsRNA complex: The chitosan-dsRNA complexes were all negatively charged, with potentials between −7 mV and −18 mV, and sizes between 40 μm and 90 μm. After the drying, the size of all the complexes was about 170 μm.

TABLE 3
dsRNA-chitosan complex parameters

| | % dsRNA in | Zeta | Wet | Dry |
|---|---|---|---|---|
| | dry complex | potential | complex | complex |
| | (w/w) | (mV) | Size (μm) | size (μm) |
| GLY3PD | 64.70 | −15 | 89 | 176 |
| PTP2 | 55.10 | −17.9 | 53 | 173 |
| EhSPW1 | 55.90 | −9 | 36 | 170 |
| ABCG7 | 50.80 | −7 | 43 | 167 |

### Challenge Test Results

The overall results are presented in Table 4 and FIGS. 2A-B.

Table 4 shows that there was significant variability in EHP copy numbers (microsporidian load) among individual shrimp, which resulted in a difficulty performing the statistical normality test. (Without subscribing to a single hypothesis, this variation in EHP copy number might be due to the genetic diversity of the animals which were obtained from local farms in which shrimp in one crop are often derived from multiple families).

However, the results indicate that administration of dsRNA targeting EHP genes dramatically reduced the microsporidian load at both 7 and 12 days post-cohabitation, whether via intramuscular injection (Treatment 3) or feeding (Treatments 1 and 2). Further, using the Kruskal-Wallis test for non-parametric data, a significant difference (P<0.05) in EHP loads was discerned at day 7 post-cohabitation (with infected shrimp) in the group fed with Feed B (Treatment 2) when compared to the other three treatments (p=0.019). On day 12 post-cohabitation, a significantly lower EHP load was observed in the group injected with treatment 3 (p=0.002). FIGS. 2A and 2B shows a graphic representation of the values in Table 4.

TABLE 4
The average levels of EHP loads in 3 replicates of
each treatment at days 7 and 12 post-cohabitation.

| | EHP copies/50 ng DNA (mean ± SE) | |
|---|---|---|
| Treatment | Day7 | Day12 |
| Positive | 255,918.44 ± 139,762.57^{a} | 207,112.63 ± 131,479.56 ^{a} |
| (Treatment 4) | | |
| Feed A | 13,091.76 ± 13,086.31 ^{ab} | 83,704.23 ± 43,890.43 ^{a} |
| (Treatment 1) | | |
| Feed B | 553.66 ± 500.01 ^{b} | 9,394.60 ± 6,018.88 ^{ab} |
| (Treatment 2) | | |
| dsRNA-B | 8,565.60 ± 6,773.34 ^{ab} | 6,792.75 ± 6,729.33 ^{b} |
| (Treatment 3) | | |
| p-value | 0.019 | 0.002 |

Taken together, these results indicate that chitosan:RNA particles prepared according to the methods of the present invention, bearing gene silencing RNA molecules targeting microsporidian parasites are capable of delivering RNAi agents effective in protecting farmed shrimps from infection with Enterocytozoon hepatopenaei when mixed with feed for oral delivery.

### Example II: Effective Prevention of EHP Infection Targeting Multiple Genes with RNAi

### Bioinformatic Analysis and Selection of Gene Targets

In addition to targeting genes responsible for cell invasion (PTP2, EhSWP1) and EHP metabolism (ABCG7 and Gly3PD) identified above, other essential EHP genes, responsible for spore wall formation (SWP3 and SWP7) and cell invasion (endochitinase and spore wall protein OQS54254) were targeted, in RNAi vaccines directed to multiple silencing targets. dsRNA sequences for the targeted genes were designed by optimization RNAi target-sites by ranking sequences according to their predicted specificity, efficiency and complexity using the online service https://www(dot)dkfz(dot)de/signaling/e-rnai3/and were synthesized commercially by from Genolution http://genolution(dot)co(dot)kr/agrorna/service-overview/.

### Spore Wall Protein 3—SWP3

SWP3, a protein abundant in mature EHP spores, is predominantly located in the endospore wall. In the proliferative stage of EHP, it was observed on the plasma membrane. SWP3 may play a significant role in spore proliferation or maturation. The presence of putative glycosylation and phosphorylation sites in the amino acid sequence may also be important for host-parasite interactions.

### Spore Wall Protein 7—SWP7

SWP7 shares homology with the sporoplasm surface protein 1 of E. hellem (EhSSP1). EhSSP1, a recently discovered sporoplasm surface protein, can attach to host cell mitochondria through a host voltage-dependent anion channel protein (VDAC) during the invasion synapse, thereby playing a crucial role in the final stages of invasion.

### Endochitinase

The EHP spore is enriched with chitin, which contributes to its resilience and survival within the host's tissues. Considering the essential role of chitin in EHP spore formation and its protective function, the Endochitinase enzyme involved in chitin synthesis represents a potential target for controlling or silencing the parasite. Remarkably, the Endochitinase enzyme does not show homology to the corresponding enzyme found in shrimp, which implies that it is unique to EHP and likely plays a crucial role in the parasite's life cycle.

### New Spore Wall Protein—OQS54254

This spore wall protein has been identified as a protein involved in the host-parasite interaction.

### Experimental Methods

### Treatment Preparations

Synthetic dsRNAs were purchased from Genolution Inc, Republic of Korea.

### Injection Preparation

dsRNA molecules were purified using isopropanol precipitation and quantified by RP-HPLC. The injection cocktails were prepared by mixing the four molecules to the final concentration of 1 μg of dsRNA per 1 gram shrimp (of each molecule), in total equaling 6 μg dsRNA per 1 gram of shrimp. Table 5 shows the genes targeted in each of the 4 vaccine cocktails.

TABLE 5
Vaccine cocktails composition

| Vaccine | Vaccine | Vaccine | Vaccine |
|---|---|---|---|
| cocktail 1 | cocktail 2 | cocktail 3 | cocktail 4 |
| PTP2 | SWP3 | PTP2 | PTP2 |
| SWP1 | SWP7 | SWP1 | SWP1 |
| GLY3PD | OQS54254 | SWP3 | OQS54254 |
| ABCG7 | Endochitinase | SWP7 | Endochitinase |
| SWP3 | GLY3PD | OQS54254 | GLY3PD |
| SWP7 | ABCG7 | Endochitinase | ABCG7 |

Previously untested dsRNA molecules: SWP3, SWP7, Endochitinase, OQS54254 (Bolded)
Previously tested dsRNA molecules: PTP2. SWP1, GLY3PD, ABCG7 (Italic)

Previously untested dsRNA molecules: SWP3, SWP7, Endochitinase, OQS54254 (Bolded)

Previously tested dsRNA molecules: PTP2. SWP1, GLY3PD, ABCG7 (Italic)

### Challenge Test

The challenge test was conducted at the University of Arizona (UAZ). During this test, the effect of 4 different vaccine cocktails on EHP propagation was evaluated by injection. P. vannamei weighing 0.8-1.2 g were stocked into eighteen (18) 90 L tanks. Each tank was stocked with 20 shrimp per tank and each treatment was tested in triplicate. All aquaria were fed a commercial shrimp diet twice a day at 7.5% body weight daily for the duration of the challenge. EHP infection was initiated by feeding the challenged tanks with the infected approximately 2 gr per aquarium EHP tissues.

On day 0, all tanks designated for the EHP challenge were fed EHP-infected hepatopancreas tissue. To ensure tissue consumption, tissue feeding replaced the commercial shrimp diet for the first feeding of the day. On day 1, each P. vannamei requiring vaccination (“treatment”) was individually injected with 50 μl of the required vaccine cocktail and returned to its respective tank (see Table 6). Shrimp were fed and observed twice daily for the duration of the study.

On day 15 of the EHP challenge, 15 shrimp from each tank were randomly selected and each had their hepatopancreas excised. Three pools containing 5 hepatopancreata were frozen at −80° C. and submitted for PCR analysis. In total, 9 PCR results were obtained from each treatment.

TABLE 6
Experimental Design

| | | | | EHP |
|---|---|---|---|---|
| Tank # | Shrimp # | Tank Designation | Treatment | Challenge |
| 1-3 | 20 per tank | Mock | None | NO |
| 4-6 | 20 per tank | Negative Control | None | YES |
| 7-9 | 20 per tank | EHP Challenge | Vaccine 1 | YES |
| 10-12 | 20 per tank | EHP Challenge | Vaccine 2 | YES |
| 13-15 | 20 per tank | EHP Challenge | Vaccine 3 | YES |
| 16-18 | 20 per tank | EHP Challenge | Vaccine 4 | YES |

### Results

On day 15 of the EHP challenge, 15 shrimp from each tank were randomly selected and three pools containing 5 hepatopancreata were submitted for PCR analysis. In total, 9 PCR results were obtained from each treatment. The results were converted to EHP copy number, and the average copy number was calculated for each treatment.

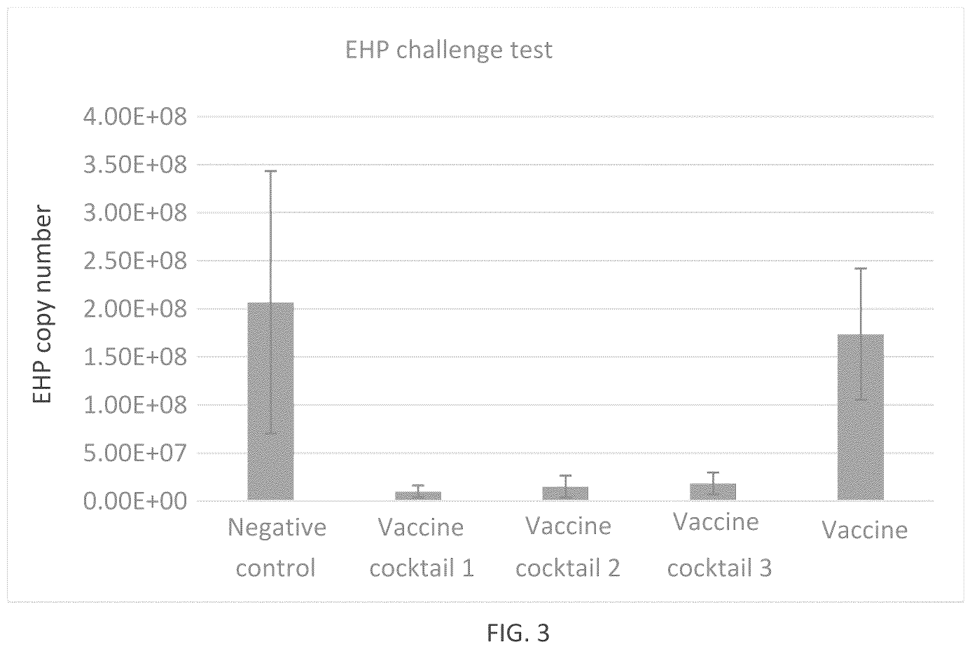

Vaccine 1, composed of 6 dsRNA molecules that targeted PTP2, SWP1, SWP3, SWP7, GLY3PD and ABCG7 genes, was found to have the greatest effect on EHP propagation and EHP copy number. In contrast, vaccine 4, targeting PTP2, SWP1, GLY3PD, ABCG7 (which were already tested in the previous trial), and two new dsRNA molecules OQS54254 and endochitinase, had a negligible effect on EHP propagation (Table 7 and FIG. 3).

Based on these results, and without subscribing to a single hypothesis, it seems that dsRNA molecules that target OQS54254 and endochitinase genes may have a negative effect on EHP inhibition while dsRNA molecules that target SWP3 and SWP7 genes may have a strong positive effect on the inhibition of EHP.

TABLE 7
The effect of vaccine cocktails on the EHP load after 15 days trial

| Pool # | Negative control | Vaccine 1 | Vaccine 2 | Vaccine 3 | Vaccine 4 |
|---|---|---|---|---|---|
| 1 | 5.40E+06 | 5.34E+07 | 2.86E+06 | 9.29E+06 | 5.58E+08 |
| 2 | 5.28E+08 | 3.43E+07 | 1.11E+08 | 1.10E+08 | 1.73E+05 |
| 3 | 4.90E+07 | 0 | 1.10E+07 | 8.47E+06 | 2.89E+06 |
| 4 | 0 | 0 | 9.41E+06 | 1.37E+06 | 0 |
| 5 | 6.65E+03 | 0 | 0 | 3.43E+07 | 8.87E+06 |
| 6 | 0 | 0 | 0 | 0 | 4.07E+05 |
| 7 | 1.57E+05 | 0 | 0 | 0 | 4.12E+08 |
| 8 | 7.07E+06 | 0 | 0 | 0 | 2.91E+08 |
| 9 | 1.27E+09 | 0 | 0 | 0 | 2.88E+08 |
| Average copy | 2.07E+08 | 9.74E+06 | 1.49E+07 | 1.82E+07 | 1.73E+08 |
| number | | | | | |
| % of reduction | 0 | 95 | 93 | 91 | 16 |

It is the intent of the applicant(s) that all publications, patents and patent applications referred to in this specification are to be incorporated in their entirety by reference into the specification, as if each individual publication, patent or patent application was specifically and individually noted when referenced that it is to be incorporated herein by reference. In addition, citation or identification of any reference in this application shall not be construed as an admission that such reference is available as prior art to the present invention. To the extent that section headings are used, they should not be construed as necessarily limiting. In addition, any priority document(s) of this application is/are hereby incorporated herein by reference in its/their entirety.

1. A composition comprising chitosan and at least one type of RNA molecule comprising a nucleic acid sequence at least 50 bases in length, wherein said RNA molecule is capable of silencing expression of a gene of a microsporidian parasitic in a farmed aquatic animal when administered to said farmed aquatic animal, wherein said microsporidian is Enterocytozoon hepatopenaei (EHP) and wherein said farmed aquatic animal is a farmed crustacean.

2. (canceled)

3. The composition of claim 1, wherein said RNA molecule comprises one or more of the following:
(a) at least one sequence capable of binding through complementary base pairing to an EHP target mRNA molecule;
(b) at least one sequence having at least 90% sequence identity to an EHP target mRNA molecule;
(c) at least one sequence at least partially complementary to an EHP target mRNA molecule;
(d) at least one sequence identical to at least 21 contiguous bases of an EHP target mRNA molecule.

4. The composition of claim 3, wherein said partially complementary sequence or sequence capable of binding through complementary base pairing to said EHP target mRNA is complementary to a sequence of said EHP target mRNA at least 50, at least 75, at least 100, at least 150, at least 200, at least 250, at least 400, at least 500, at least 600, at least 750 bases in length.

5. The composition of claim 3, wherein said at least one RNA molecule comprising at least one sequence identical to at least 21 contiguous bases of an EHP target mRNA molecule is identical to at least 50, at least 75, at least 100, at least 150, at least 200, at least 250, at least 400, at least 500, at least 600, at least 750 bases of said EHP target mRNA molecule.

6. The composition of claim 1, wherein said at least one type of RNA molecule is a single stranded RNA (ssRNA) or a double-stranded RNA (dsRNA).

7. (canceled)

8. The composition of claim 1, wherein said gene is selected from the group consisting of the EHP polar tube protein 2 (PTP-2) gene, the EHP Heparin binding protein (EhSWP1) gene, the EHP ATP binding cassette (ABCG7) gene, the EHP Glyceraldehyde-3-phosphate dehydrogenase type 1 (Gly3PD) gene, the EHP ADP-ATP carrier protein gene, the EHP ADP-ATP carrier protein 4 gene, the EHP Glucose-6-phosphate dehydrogenase (ZWF1) gene, the EHP plasma membrane transporter gene, the EHP spore wall protein 3 (SWP3) gene, the EHP spore wall protein 7 (SWP7) gene, the EHP Endochitinase gene and the EHP new spore wall protein (OQS54254) gene.

9. The composition of claim 3, wherein:
i) said RNA molecule comprising at least one sequence capable of binding through complementary base pairing to an EHP target mRNA molecule or comprising at least one sequence at least partially complementary to an EHP target mRNA molecule comprises a nucleic acid sequence complementary to a nucleic acid sequence selected from the group consisting of SEQ ID NOs: 1-16, and/or
ii) said RNA molecule comprising at least one sequence having at least 90% sequence identity to an EHP target mRNA molecule or at least one sequence identical to at least 21 contiguous bases of an EHP target mRNA molecule comprises:
(a) a nucleic acid sequence identical to at least 21 contiguous bases of a nucleic acid sequence selected from the group consisting of SEQ ID NOs: 5-8, 10 and 12, or
(b) a nucleic acid sequence complementary to a nucleic acid sequence selected from the group consisting of SEQ ID NOs: 5-8, 10 and 12.

10. (canceled)

11. The composition of claim 3, comprising at least one additional RNA sequence capable of directing cleavage of an EHP target mRNA molecule.

12. The composition of claim 3, comprising at least one additional sequence capable of binding through complementary base pairing to an EHP target mRNA molecule or comprising at least one additional sequence at least partially complementary to an EHP target mRNA molecule, and wherein said EHP target mRNA molecule has a nucleic acid sequence selected from the group consisting of SEQ ID NOs: 1-16.

13. (canceled)

14. The composition of claim 12, wherein said EHP target mRNA molecule has a nucleic acid sequence selected from the group consisting of SEQ ID NOs: 5-8, 10 and 12.

15. The composition of claim 1, wherein said chitosan is 75-100% deacetylated low molecular weight chitosan and wherein the chitosan:RNA mass ratio (w/w) of said particle is in the range of 0.3-0.9.

16. The composition of claim 1, wherein the chitosan:RNA ratio is in the range of 0.45-0.55 and the molecular weight of said chitosan is in the range of 20-30 kDa.

17. The composition of claim 1, formulated with farmed crustacean food as a nutraceutical composition.

18. The composition of claim 17, wherein the RNA content (w/w) of said nutraceutical composition is in the range of 0.01-10% or 0.1%-5%.

19. (canceled)

20. A method for producing a chitosan-RNA composition comprising RNA capable of silencing expression of a gene of a microsporidian parasitic in a farmed aquatic animal, the method comprising:
(i) rapidly adding a solution of said RNA to a volume of 50-100% deacetylated low molecular weight chitosan solution, wherein said chitosan solution comprises 2-20 mg/ml low molecular weight chitosan in 0.01 M-0.1 M citric acid,
(ii) desalting and concentrating the chitosan-RNA complex solution, and
(iii) drying the desalted, concentrated chitosan-RNA complex solution,
thereby producing the chitosan-RNA composition,
wherein said microsporidian is Enterocytozoon hepatopenaei (EHP) and wherein said farmed aquatic animal is a farmed crustacean.

21-22. (canceled)

23. The method of claim 20, wherein said citric acid is 0.02 M and said RNA solution comprises 0.2-10 mg/ml RNA in water.

24-27. (canceled)

28. A method of treatment or prevention of EHP infection in a farmed crustacean, the method comprising feeding the farmed crustacean an effective amount of the composition of claim 1, thereby treating or preventing the EHP infection.

29. (canceled)

30. The method of claim 28, wherein said farmed crustaceans are selected from the group consisting of Shrimp, Prawns, Crabs, Lobsters and Crayfishes.

31. The method of claim 30, wherein said farmed crustaceans are shrimp or prawns.

32-34. (canceled)
